(19) United States
(12) Patent Application Publication
(10) Pub. No.: US 2007/0082327 A1
(43) Pub. Date: Apr. 12, 2007

(54) Cellular phenotype

(75) Inventors: Cynthia Lynn Adams, San Carlos, CA (US); Reginald Norman de la Rosa, Concord, CA (US); Shyamlal Ramchandani, San Fracisco, CA (US); Penelope Chua, San Francisco, CA (US)

Correspondence Address: BEYER WEAVER LLP, P.O. BOX 70250, OAKLAND, CA, 94612-0250, US

(73) Assignee: Cytokinetics, Inc. A Delaware Corporation

(21) Appl. No.: 11/518,500

(22) Filed: Sep. 8, 2006

Related U.S. Application Data
(60) Provisional application No. 60/715,502, filed on Sep. 9, 2005.

(51) Int. Cl. C12Q 1/00; G01N 33/574; G06F 19/00; C12N 5/06

(52) U.S. Cl. 435/4; 435/7.23; 435/325; 702/19

(57) ABSTRACT

Phenotypes and the cells that exhibit those phenotypes are described. The phenotype may be established as a “snapshot” of the cells at a particular time or it may be established as a variation in features over time, or as some combination of these “static” and “dynamic” characterizations. The phenotype may be characterized by at least the following features: mitotic arrest characterized (i) chromosomes well-aligned at the metaphase plate and (ii) chromosome residence time at the metaphase plate substantially longer than that of a control cell or cell population. The phenotype may be further characterized by: during interphase the cell or population of cells exhibits a phenotype that is substantially similar to that of the interphase cells of the control cell or cell population.

### CROSS REFERENCE TO RELATED APPLICATION

This application claims benefit under 35 USC § 119(e) to U.S. Provisional Patent Application No. 60/715,502, filed Sep. 9, 2005 and titled “CELLULAR PHENOTYPE,” which is hereby incorporated by reference.

### BACKGROUND

This invention relates to particular cellular phenotypes and to the cells and populations of cells that exhibit such phenotypes. The invention also relates to methods, apparatus, and computer program products that identify and/or make use of the phenotypes.

It is often desirable to characterize a cell or cell population by its phenotype. A cell's phenotype may change when exposed to a new stimulus or a change in the level of exposure to such stimulus. A given cell line may exhibit one phenotype when exposed to a particular compound and a different phenotype when exposed to a related compound. Temperature, culture conditions, exposure time, concentration and a number of other parameters can also influence the phenotype of a cell line. In addition, a compound may produce different phenotypes in different cell lines.

Certain phenotypes are manifestations of a stimulus′ mechanism of action. As such they can help identify the mechanism of action of a stimulus under investigation such as a drug candidate. Hence, studies of phenotypic variation are valuable in drug discovery research. Specifically, a drug candidate may be characterized by its ability to elicit a particular phenotype, which indicates activity against a particular cellular target. In addition, certain phenotypic variations may indicate that a candidate has a potential side effect. When a candidate elicits a phenotypic change unrelated to the relevant target, it may be an indication that the candidate has a side effect. For additional discussion of how phenotypes are used in drug discovery, see U.S. patent application Ser. No. 10/621,821, filed Jul. 16, 2003, by Kutsyy et al., and titled “METHODS AND APPARATUS FOR INVESTIGATING SIDE EFFECTS,” which is incorporated herein by reference for all purposes.

The potential of phenotypic studies has not been realized. Some phenotypes associated with particular mechanisms of action, side effects, etc. have yet to be characterized or even observed. New avenues of cell biology research may yield novel phenotypes having utility in drug discovery and other areas.

### SUMMARY

Generally, this invention relates to specific phenotypes and the cells that exhibit these phenotypes. Note that the concept of a “phenotype” includes characterizations of morphological features (size, shape, distribution/concentration of cell components, etc.), as well as the gross features of a cell population (motility, arrest in a particular stage of the cell cycle, growth and division rate, death rate, etc.). The phenotype may be established as a “snapshot” of the cells at a particular time or it may be established as a variation in features over time, or as some combination of these “static” and “dynamic” characterizations. It may also be defined in terms of changes that occur in response to various levels or doses of a particular stimulus. In such cases, the phenotype is represented, at least in part, as a stimulus-response path. Further, the phenotype may be defined over multiple cell lines, with some lines showing a greater susceptibility to particular phenotypic features than other cell lines.

One aspect of the invention provides a phenotype embodied in cell or a population of cells. The phenotype is referred to as the mp2 phenotype in this application. The term mp2 describes certain characteristics of the phenotype and is not limited to any particular type of cell line. The mp2 phenotype of this invention may be characterized by at least the following features: mitotic arrest characterized by (i) chromosomes well-aligned at the metaphase plate, and (ii) chromosome residence time at the metaphase plate substantially longer than that of a control cell or cell population. In some embodiments, chromosome residence time in a well-aligned metaphase plate is at least about 3-10 times longer than control. According to various embodiments, mitotic arrest may last from about 3 to 24 hours. Further examples of features that may be used to characterize the mp2 phenotype include: (b) chromosomes that congress normally to the metaphase plate, and (c) during interphase, the cell or population of cells exhibits a phenotype that is substantially similar to that of the control cell or cell population. Examples of other features that may be used to characterize the mp2 phenotype include the following: (d) a higher percentage of the cells in the cell population that die prematurely in comparison to the control cell or cell population, (e) stable microtubule-kinetochore attachment and/or alignment at the metaphase plate and (f) a high percentage of cells in the cell population that exhibit the other characteristics of the phenotype.

In addition, stimuli that produce the mp2 phenotype do so selectively in some cells, or at least do so to a significantly lesser degree in the others. For example, normal (non-tumor) cell type IMR-90 is less susceptible to stimuli that produce the mp2 phenotype than tumor cell types SKOV3, A549, MV522 or HT29.

Another aspect of the invention pertains to particular eukaryotic cells (e.g., mammalian cells) or cell populations that exhibit the mp2 phenotype. These cells or populations will possess at least the features identified above. Typically, the mp2 phenotype will be produced by applying a stimulus to the cell or cell population that does not initially exhibit the mp2 phenotype. The stimulus induces a transformation to produce the mp2 phenotype. In some embodiments, applying the stimulus comprises administering a compound to the cells or population(s).

The invention also pertains to methods and apparatus used to investigate, characterize, or otherwise quantify, an effect under investigation for its ability to produce an mp2 phenotype of this invention. One method aspect of the invention produces a transformation in the phenotype of a cell or cell population by (a) exposing the cell or cell population to a stimulus; and (b) allowing the stimulus to interact with the cell or cell population in a manner that transforms the cell or cell population to give rise to a phenotype having at least some of the features described above. The method may further involve (c) imaging the cell or cell population to capture features that characterize the phenotype of the cell or cell population; and (d) analyzing the image to determine whether the cell or cell population exhibits the phenotypic features specified in (b), to thereby determine whether the compound produces the transformation. In many cases, the stimulus involves exposure to a particular compound or group of compounds.

Apparatus of the invention may include devices for providing cells (e.g., cell cultures in multi-well plates), delivering stimulus to the cells (possibly in carefully metered amounts), imaging the cells before, during, and/or after exposure to the stimulus, analyzing the image, or any combination of such devices.

Another aspect of the invention provides a method of characterizing a cell or a cell population based on phenotype. The method may be characterized by the following sequence: (a) receiving data characterizing the phenotype of the cell or cell population; (b) analyzing the data to determine whether the cell or cell population possesses some or all of the phenotypic features identified above; and (c) characterizing the cell or cell population as having a mp2 phenotype when the cell or cell population is found to possess at least a requisite set of the features specified above. Note that when phenotypic data is collected across multiple cell lines, the information can be used to characterize the specificity of a treatment.

Another aspect of the invention pertains to computer program products including machine-readable media on which are stored program instructions for implementing at least some portion of the methods described above. Any of the methods of this invention may be represented, in whole or in part, as program instructions that can be provided on such computer readable media. In addition, the invention pertains to various combinations of data and associated data structures generated and/or used as described herein.

These and other features and advantages of the present invention will be described in more detail below with reference to the associated figures.

### BRIEF DESCRIPTION OF THE DRAWINGS

The patent or application file contains at least one drawings executed in color. Copies of this patent or patent application publication with color drawing(s) will be provided by the Office upon request and payment of the necessary fee.

FIG. 1a shows representative time-lapse images of GFP-histone 2B in SKOV3 cells undergoing normal mitosis. The images were taken at 3 minute intervals using 60× magnification. The numbers on each panel represent the number of hours that have elapsed from prometaphase.

FIG. 1b shows representative time-lapse images of SKOV3 cells in the presence of an mp2 stimulus compound and exhibiting the mp2 phenotype according to certain embodiments. Images were taken at 3 minute intervals using 60× magnification. The numbers on each panel represent the number of hours that have elapsed from prometaphase.

FIG. 1c shows kinetochore and microtubule staining in SKOV3 cells treated in the presence of DMSO (control) and an mp2 stimulus compound.

FIG. 2 shows example time-lapse images of a SKOV3 cell in the presence of an mp2 stimulus and exhibiting the mp2 phenotype according to certain embodiments. Progression of the cell from interphase just prior to chromosome condensation, to mitotic arrest, to decondensation, and then to apoptosis. Elapsed time in hours is shown below each image. The images were taken at 15 minute intervals at 10× magnification.

FIG. 3a is a bar graph showing the percentages of 20 random cells tracked to assess their fate: complete mitosis, death from mitosis, decondensation (uncertain fate) and death from decondensation. Data was taken from SKOV3 cells in the presence of mp2 stimulus compounds and exhibiting mp2 phenotypes as compared to cells treated with Taxol (paclitaxel) and rice phenotype stimulus compounds (both mitotic inhibitors) and well as with DMSO (control).

FIG. 3b is a bar graph showing the percentages of 20 random cells tracked to assess their fate: complete mitosis, death from mitosis, decondensation (uncertain fate) and death from decondensation. Data was taken from A549 cells in the presence of mp2 stimulus compounds and exhibiting mp2 phenotypes as compared to cells treated with Taxol (paclitaxel) and rice phenotype stimulus compounds (both mitotic inhibitors) and well as with DMSO (control).

FIG. 4 is graph showing how increases in the mitotic index statistic in SKOV3 cells (which measures a compound's ability to cause mitotic arrest) varies as a function of a phenotypic “distance” from a normal interphase phenotype in HUVEC cells at the same concentration for phenotypes of this invention and certain other phenotypes. The data is from representative compounds including compounds capable of inducing the mp2 phenotype, Taxol, other mitotic inhibitors and control.

FIG. 5 presents MTS dose response curves for a KSP inhibitor (an inhibitor of mitotic kinesins) and an mp2 producing compound in a tumor cell line (MV522) and a normal cell line (IMR90).

FIG. 6a is a graph depicting area under the average MTS dose response curve data for normal (IMR90) and a panel of tumor cell lines (SKOV3, A549, HT29, MV522) treated with various mitotic inhibitors.

FIG. 6b presents representative images from time-lapse movies of GFP-Histone 2B expressing SKOV3, A549, HT29, MX1 and HeLa cells prior to adding an mp2 stimulus compound (top images) and then 20 hours after the addition of the mp2 stimulus compound (bottom images). The images were collected every 15 minutes at 10× magnification.

FIGS. 7A and 7B present G150, TGI and LC data for 60 cancer cell lines treated with an mp2 inducing compound. The data was collected and determined by the National Cancer Institute (NCI) using an MTT reporting assay.

FIGS. 7C through 7K present percentage growth data for 60 cancer cell lines treated with various concentrations of an mp2 inducing compound. The data is presented by type of cancer cell line.

FIG. 8 presents graphs depicting the results of clonogenic viability assays of MV522 cells treated with Taxol and compounds that produce the mp2 phenotype. Images of the images quantified for the graph are also depicted.

FIG. 9 is a flow chart illustrating an embodiment of a general method employed to quantitatively determine whether a stimulus gives rise to the mp2 phenotype.

FIG. 10 is a flow chart illustrating cell sample preparation activities of the method illustrated by FIG. 9 in greater detail.

FIG. 11 is a flow chart illustrating image capture and processing activities of the method illustrated in FIG. 9 in greater detail.

FIG. 12 is a schematic block diagram of an embodiment of an image capture and image processing system suitable for carrying out some of the activities illustrated in FIG. 11.

FIG. 13 is a simplified block diagram of a computer system that may be used to implement various aspects of this invention, including characterizing cellular phenotypes, determining whether a given phenotype is a mp2 phenotype, and calculating distances between control and test phenotypes using “signatures” of those phenotypes.

### DESCRIPTION OF A PREFERRED EMBODIMENT

I. Introduction

As indicated, this invention pertains to phenotypes that were not previously observed. They may arise from a unique type of disruption to the mitotic apparatus in eukaryotic cells, although the invention is not limited to phenotypes arising from any particular stimulus. The phenotypes of this invention are referred to herein as “mp2 phenotypes.” mp2 phenotypes are generally characterized by mitotic arrest, more specifically by an unnaturally long residence time of chromosomes at the metaphase plate often with failure to progress through normal mitosis thereafter. The mitotic arrest of the mp2 phenotypes may be further characterized by most or all of the chromosomes are well aligned at the metaphase plate and stable microtubule-kinetochore alignment. However, it is commonly found that at least one chromosome pair fails to stably align with the metaphase plate. Typically, though not necessarily, all of these features are present in a phenotype of this invention. Further features that are typically, though not necessarily, present in phenotypes of this invention are a substantially unperturbed interphase phenotype, and chromosomes that fail to reach an anaphase state (i.e., chromosomes that fail to separate and move toward the poles of the spindle). After a prolonged mitotic arrest, the condensed chromosomes and microtubules typically become disorganized. Another common feature of cells exhibiting the mp2 phenotype is decondensation of the chromosomes and/or death (usually by apoptosis) following this mitotic arrest. Treatments that produce the phenotype do so in a unique cell-line specific pattern. Another interesting feature is that the treatments that produce the phenotype are more efficient at killing tumor cells than some known mitotic inhibitors.

Note that characteristics of the mp2 phenotype are defined with respect to a control cell or population of cells, which has not been exposed to a stimulus that produces the novel phenotype. Aside from exposure to such stimulus, the control and the test cells should be similar in terms of genotype and history (source, culturing, environment influences, etc.).

Any given cell that exhibits the features identified above may be characterized as having an mp2 phenotype of this invention. However, a population of cells may also be said to possess the mp2 phenotype if some number or a percentage of its member cells exhibit the above features (when compared to a control population that have not been exposed to a stimulus that produces the mp2 phenotype). For example, the phenotype may be present if on average the members of the population exhibit the features. Further, it has been observed that certain interesting phenotypic characteristics typically occur only in a fraction of a cell population exhibiting the mp2 phenotype. An example is death directly from mitosis.

As explained below, phenotypes of this invention may be identified by eye, manual measurement, automated measurement and analysis, etc. However, certain specific aspects of this invention pertain to automated image analysis techniques that identify phenotypes of this invention. Such techniques may make use of markers for cellular components that assume interesting structures during mitosis and interphase states. Examples of such components include histones, DNA, tubulin, Golgi apparatus and certain other cytoskeletal components such as actin.

The mp2 phenotype may be generated by any of a number of different stimuli. It has been found that exposure to a particular class of compounds generates the heretofore unknown phenotype. These compounds include, for example, those described in U.S. Provisional Patent Application No. 60/622,282, filed Oct. 25, 2004, which is incorporated herein by reference for all purposes.

II. Definitions

Some of the terms used herein are not commonly used in the art. Other terms may have multiple connotations in the art. Therefore, the following definitions are provided as an aid to understanding the description herein. The invention as set forth in the claims should not necessarily be limited by these definitions.

The term “component” or “component of a cell” refers to a part of a cell having some interesting property that can be characterized by image analysis to derive biologically relevant information. General examples of cell components include biomolecules and subcellular organelles. Specific examples of biomolecules that can serve as cell components include specific proteins and peptides, lipids, polysaccharides, nucleic acids, etc. Sometimes, the relevant component will include a group of structurally or functionally related biomolecules. Alternatively, the component may represent a portion of a biomolecule such as a polysaccharide group on a protein, or a particular subsequence of a nucleic acid or protein. Collections of molecules such as micells can also serve as cellular components for use with this invention. And subcellular structures such as vesicles and organelles may also serve the purpose.

The term “marker” or “labeling agent” refers to materials that specifically bind to and label cell components. These markers or labeling agents should be detectable in an image of the relevant cells. Typically, a labeling agent emits a signal whose intensity is related to the concentration of the cell component to which the agent binds. Preferably, the signal intensity is directly proportional to the concentration of the underlying cell component. The location of the signal source (i.e., the position of the marker) should be detectable in an image of the relevant cells.

Preferably, the chosen marker binds specifically with its corresponding cellular component, regardless of location within the cell. Although in other embodiments, the chosen marker may bind to specific subsets of the component of interest (e.g., it binds only to sequences of DNA or regions of a chromosome). The marker should provide a strong contrast to other features in a given image. To this end, the marker may be luminescent, radioactive, fluorescent, etc. Various stains and compounds may serve this purpose. Examples of such compounds include fluorescently labeled antibodies to the cellular component of interest, fluorescent intercalators, and fluorescent lectins. The antibodies may be fluorescently labeled either directly or indirectly.

The term “stimulus” refers to something that may influence the biological condition of a cell. Often the term will be synonymous with “agent” or “manipulation” or “treatment.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

A particularly important class of stimuli in the context of this invention is chemical compounds, including compounds that are drugs or drug candidates and compounds that are present in the environment. The biological impact of chemical compounds is manifest as clear phenotypic changes such as those producing phenotypes of this invention. Related stimuli involve suppression of particular targets by siRNA or other tool for preventing or inhibiting expression.

The term “phenotype” generally refers to the total appearance and behavior of a cell or multi-cellular organism. The phenotype results from the interaction of an organism's genotype and the environment. Cellular phenotypes may be defined in terms of various qualitative and quantitative features. These features may be captured and stored in images and in numeric and/or symbolic representations in processing systems (e.g., computers) and data storage media (whether or not directly associated with a computer system). For certain embodiments of this invention, the phenotype is a characteristic of a population of similarly situated cells (having a common environment and/or history of interactions with the environment). Thus, the phenotype may be manifest by particular visible features and/or behaviors that vary depending upon the state of the cell. For example, a phenotype may be manifest by one feature while in the mitotic portion of the cell cycle and a different, even unrelated, feature while in interphase portion of the cell cycle.

Often a particular phenotype can be correlated or associated with a particular biological condition or mechanism of action resulting from exposure to a stimulus. Generally, cells undergoing a change in biological conditions will undergo a corresponding change in phenotype. Thus, cellular phenotypic data and characterizations may be exploited to deduce mechanisms of action and other aspects of cellular responses to various stimuli.

A selected collection of data and characterizations that represent a phenotype of a given cell or group of cells is sometimes referred to as a “quantitative cellular phenotype.” This combination is also sometimes referred to as a phenotypic fingerprint or just “fingerprint.” The multiple cellular attributes or features of the quantitative phenotype can be collectively stored and/or indexed, numerically or otherwise. The attributes are typically quantified in the context of specific cellular components or markers. Measured attributes useful for characterizing an associated phenotype include morphological descriptors (e.g., size, shape, and/or location of the organelle), cell count, motility, composition (e.g., concentration distribution of particular biomolecules within the organelle), and variations in the degree to which different cells exhibit particular features. Often, the attributes represent the collective value of a feature over some or all cells in an image (e.g., some or all cells in a specific well of a plate). The collective value may be an average over all cells, a mean value, a maximum value, a minimum value or some other statistical representation of the values.

The quantitative phenotypes may themselves serve as individual points on “response curves.” A phenotypic response to stimulus may be determined by exposing various cell lines to a stimulus of interest at various levels (e.g., doses of radiation or concentrations of a compound). In each level within this range, the phenotypic descriptors of interest are measured to generate quantitative phenotypes associated with levels of stimulus.

The term “path” or “response curve” refers to the characterization of a stimulus at various levels. For example, the path may characterize the effect of a chemical applied at various concentrations or the effect of electromagnetic radiation provided to cells at various levels of intensity or the effect of depriving a cell of various levels of a nutrient. Mathematically, the path is made up of multiple points, each at a different level of the stimulus. In accordance with this invention, each of these points (sometimes called signatures) is preferably a collection of parameters or characterizations describing some aspect of a cell or collection of cells. Typically, at least some of these parameters and/or characterizations are derived from images of the cells. In this regard, they represent quantitative phenotypes of the cells. In the sense that each point or signature in the path may contain more than one piece of information about a cell, the points may be viewed as arrays, vectors, matrices, etc. To the extent that the path connects points containing phenotypic information (separate quantitative phenotypes), the path itself may be viewed as a “concentration-independent phenotype.” The generation and use of stimulus response paths is described in more detail in U.S. patent application Ser. No. 09/789,595 (U.S. Patent Publication No. 20020155420), filed Feb. 20, 2001 naming Vaisberg et al., and titled, “CHARACTERIZING BIOLOGICAL STIMULI BY RESPONSE CURVES,” and U.S. patent application Ser. No. 60/509,040, filed on Jul. 18, 2003, naming V. Kutsyy, D. Coleman, and E. Vaisberg as inventors, and titled, “Characterizing Biological Stimuli by Response Curves,” both of which are incorporated herein by reference for all purposes.

As used herein, the term “feature” refers to a phenotypic property of a cell or population of cells. As indicated, individual quantitative phenotypes (fingerprints) are each comprised of multiple features. The terms “descriptor” and “attribute” may be used synonymously with “feature.” Features derived from cell images include both the basic “features” extracted from a cell image and the “biological characterizations” (including biological classifications such as cell cycle states). The latter example of a feature is typically obtained from an algorithm that acts on a more basic feature. The basic features are typically morphological, concentration, and/or statistical values obtained by analyzing a cell image showing the positions and concentrations of one or more markers bound within the cells.

III. Phenotypic Characteristics

1. Prolonged Metaphase

In phenotypes of this invention, the cell or cells undergo mitotic arrest chiefly characterized by a prolonged metaphase as compared to a control phenotype. More specifically, the residence time of the chromosomes at the metaphase plate in cells exhibiting the mp2 phenotype is typically longer than that observed in control cells. Prolonged time in metaphase may be characterized by most or all of chromosomes aligned at the metaphase plate and/or stable microtubule-kinetochore alignment at the metaphase plate. Metaphase arrest caused by mp2 stimuli is typically at least six times, on average 20 times, and can be 40 times longer than a control division for SKOV3 cells.

As is well-known, during prometaphase, chromosomes in normal cells establish interactions with the fast-growing plus ends of microtubules via the kinetochore. The kinetochore of each sister chromatid in a chromosome is attached to microtubules arising from spindle poles. The chromosomes then undergo a series of microtuble-dependent movements that culminate in alignment at the metaphase plate, equidistant from the two spindle poles, at metaphase. This process is called “congression.” The mitotic spindle at metaphase is a dynamic, yet balanced, structure that holds the chromosomes at the metaphase plate. Although there is movement of tubulin subunits, the lengths of the kinetochore and microtubules are stable. A “checkpoint” stays on until all chromosomes have been attached to microtubules and aligned; a single unattached kinetochore is enough to keep the checkpoint on and prevent entry into anaphase. In normal cells, once the checkpoint is off, the sister chromatids separate in anaphase toward opposite poles.

In the phenotypes of this invention, the chromosomes appear to congress to metaphase and then fail to divide compared to control phenotypes. Instead, during the prolonged metaphase, the chromosomes remain in a stable alignment at the metaphase plate. Kinetochore-microtubule attachment also appears to be stable. This is in contrast to a normal, untreated cell in which the chromosomes do not appear to be arrested or ‘hang’ at the metaphase plate, but undergo congression, alignment and separation fairly rapidly. The entire mitotic cycle from prometaphase to late anaphase takes from 1-2 hours at most for most human cancer and normal cell lines, and the time spend at metaphase alignment, while difficult to measure, is significantly shorter, from 10-30 minutes.

It can be useful to employ time-lapse imaging technology to characterize the progression of chromosomes during mitosis. As described above, the phenotypes of this invention are characterized by during mitotic arrest with dynamic, yet relatively structured, DNA movements and organization. A specific example of a time-lapse experiment will now be described. Using multi-site time-lapse imaging of live cells expressing a GFP-histone2B (or other GFP-tagged histone) at low (5×-10×) or high (around 60×) magnification, the mitotic DNA progression can be observed. Cells can be kept alive in their preferred environment using an environmental chamber with heat and carbon dioxide, using for example, apparatus available for this purpose such as the ImageXpress live cell imaging system available from Axon Instruments of Union City, Calif. Many wells can be sequentially visited and images can be taken. This process can be repeated every 10-15 minutes over a course of days, if appropriate, in the presence of a compound or control conditions, until hundreds of images are collected that can be collated into movies and analyzed qualitatively or quantitatively.

The DNA aspect of the mp2 phenotype may be observed by any technique that can distinguish chromosomal material from other cellular features and background. In many cases, it is convenient to generate images of cells that have been treated with markers for DNA and/or histones. Examples of such markers include fluorescently labeled antibodies to DNA and fluorescent DNA intercalators such DAPI and Hoechst 33342 (available from Molecular Probes, Inc. of Eugene, Oreg.) and antibodies to histones such as an antibody for a phosphorylated histone, e.g., phospho-histone 3 (pH3). The histones in the nucleus become phosphorylated during mitosis and remain phosphorylated while the cell is in mitotic arrest. Therefore markers specific to phosphorylated histones will mark chromatin selectively in mitotic cells. Another option (although it does not selectively mark mitotic cells) is to use cells expressing a GFP-histone2B (or any other GFP-tagged protein that functionally co-localizes with nuclear DNA).

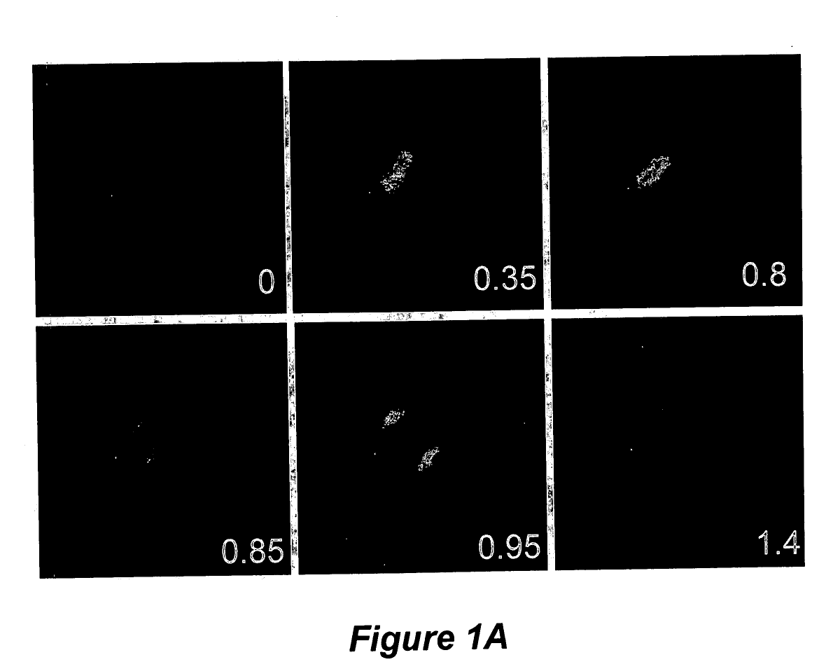

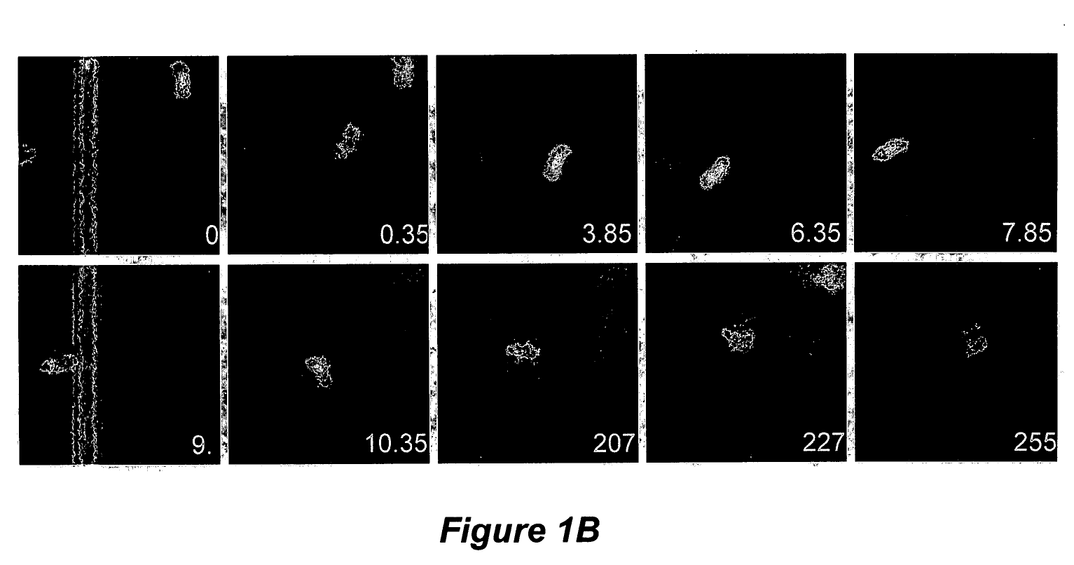

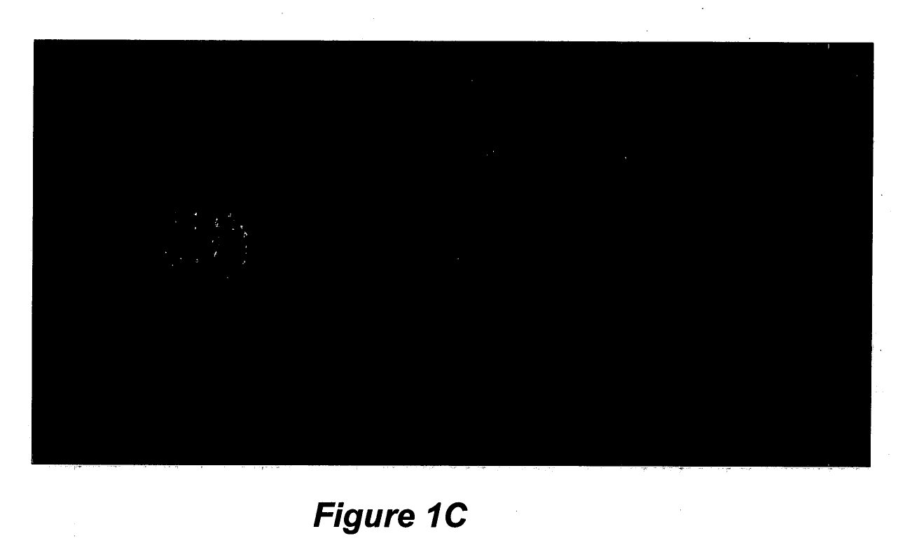

FIG. 1a shows time-lapse images of GFP-histone 2B in control SKOV3 cells moving from left to right in rows and then top to bottom. The control cells shown in FIG. 1a were treated with 0.4% DMSO during mitosis covering 2 hours. Images were taken every 3 minutes; images at 0, 0.35 hours, 0.8 hours, 0.85 hours, 0.95 hours and 1.4 hours are shown. The control montage shows normal mitotic progression of chromatin. The control cell progresses from prophase, to prometaphase (image at 0.35 hrs), to metaphase (image at 0.8 hours), to anaphase (image at 0.85 hours) and onto telophase (image at 1.4 hours).

FIG. 1b shows time-lapse images of GFP-histone 2B in SKOV3 cells treated with 150 nM of an mp2 compound moving left to right in rows and then top to bottom. As with the images of the control cells shown in FIG. 1a, images were every 3 minutes; images at 0. 0.35, 3.85, 6.35, 7.85, 9, 10, 14, 15 and 17 hours are shown. From 0.35 hrs-7.85 hours, the chromosomes appear to be aligned at the metaphase plate, as in the 0.8 hour image in FIG. 1a. However, unlike in the control cells which separate by 0.85 hours, the chromosomes remain in this organized state for a prolonged period. Specifically, at 6.35 and 7.85 hours, the chromosomes still appear to be well aligned at the metaphase plate. After 8 hours the well aligned metaphase plate become disorganized but the DNA remains condensed for another 9 hours, at which time data collected was stopped. The chromatin never segregates into daughter chromosomes in this time frame.

The prolonged time in metaphase and the failure of many cells to progress to anaphase may suggest that the cells exhibiting the mp2 phenotype either fail to complete alignment or have a defect in the metaphase to anaphase transition. Prolonged time in metaphase in the phenotypes of this invention thus encompasses prolonged time in a state in which some or most of the chromosomes have aligned at the metaphase plate, but at least one pair has not aligned. Indeed, in some cells exhibiting the phenotype, high-resolution (60×) images show one or more chromosomes pairs that do not align at the metaphase plate. These chromosomes may appear to ‘oscillate’ around the metaphase plate. (In the context of this invention, the term oscillate refers to movement to and/or from and/or along the metaphase plate, such movement not necessarily having an underlying periodicity. The movement is greater than that typically exhibited by chromosomes aligned at the metaphase plate.) However, as discussed above, most chromosomes do not exhibit oscillations at the metaphase plate. Rather, the overall appearance is of well-aligned chromosomes at the metaphase plate.

The prolonged metaphase aspect of the phenotype is not found in phenotypes produced by many types of stimulus that interfere with the mitotic apparatus, including the kinetochore. Examples of compounds that interfere with mitosis but do not produce prolonged metaphase include Taxol and the vinca alkaloids and various compounds that interact with active sites on various kinetochore associated proteins or proteins involved in pre-metaphase arrest (e.g., KSP, CENP-E, RABK6, BubR1, and Aurora (AUR1, and AUR2)). Hence, phenotypes of this invention are surprisingly easy to distinguish from phenotypes produced by such compounds.

In some embodiments, the chromosome or chromatin feature of the mp2 phenotype observed during mitosis can be presented as a multivariate signature. For example, this feature might be characterized by a signature combining the following values: (1) location of chromatin with respect to the metaphase plate during metaphase, (2) time in metaphase; and (3) failure to reach anaphase (Y or N). In this example, the resulting multivariate signature is characterized in terms of its “distance” (in multivariate phenotype space) from a control phenotype signature. Certain separation distances are associated with the mp2 phenotype of this invention. Various techniques for measuring distance in multivariate space may be used. Some are described below in the context of interphase phenotypes.

As can be seen in comparing the SKOV3 cells exhibiting the mp2 phenotype in FIG. 1b to control SKOV3 cells in FIG. 1a, the time spent in metaphase is significantly longer than control. Normal cells typically spend less than one hour in prometaphase and metaphase. In many embodiments, cells exhibiting the mp2 phenotype spend at least three times in these states as control cells. In some embodiments, the prolonged metaphase may range, on average, from three to twenty-four hours. However, the duration of the prolonged arrest is cell line dependent, as well as dependent on the stimulus.

In addition to the chromosome or chromatin feature described above, prolonged metaphase may be measured by kinetochore-microtubule location and/or alignment at the metaphase plate. As noted above, cells exhibiting the mp2 phenotype have stable kinetochore-microtubule alignment at the metaphase plate during the prolonged metaphase period. This aspect of the phenotype may be observed with any technique that can distinguish these features from other cellular features and background. In many cases, it is convenient to generate images of cells that have been treated with kinetochore and microtubule markers such as MAD2, HEC1, CREST, and securin. FIG. 1c shows kinetochore and microtubule staining in SKOV3 cells treated in the presence of DMSO (control) and an mp2 inducing compound. In some embodiments, the mp2 phenotype may also be characterized by apparent stable kinetochore-microtubule attachment.

2. Normal Congression to Metaphase Plate

As noted above, the prolonged metaphase is characterized by organized chromosome alignment and stable microtubule-kinetochore attachment. Thus, generally speaking, during the prolonged metaphase state, cells exhibiting the mp2 phenotype look like normal cells during metaphase, with duration of metaphase being the main distinguishing feature. Of course, in certain embodiments, one or more chromosome pairs do not undergo attachment and/or stable alignment.

A related feature found in many of the phenotypes of this invention is that cell or cell lines exhibit normal congression to the metaphase plate as compared to control. As described above, the term “congression” refers to microtubule-kinetochore attachment and the microtuble-dependent movements that culminate in alignment at the metaphase plate, equidistant from the two spindle poles, at metaphase.

Normal congression indicates that at least most chromosomes congress to the metaphase plate in the time and manner exhibited by control cells. In many cells exhibiting the phenotype, all but one to two pairs of chromosomes congress normally to the metaphase plate.

3. Cells Die in a Defined Manner

Most cells exhibiting the mp2 phenotype do not complete mitosis. Most of these cells ultimately die. Hence a population of cells exhibiting the phenotype will have, in comparison to a control, an unusually high proportion of cells that have died. Various techniques for identifying dead cells may be used. In the context of image analysis, cell count (or number of objects) is a useful measure of the impact of a stimulus on cell viability. At a certain length of time after a cell dies, some markers for viable cells no longer appear in images of the dead cell. Hence, in images of such markers, dead cells no longer appear as separate objects.

After mitotic arrest, the condensed chromosomes and microtubules become disorganized. In some cells exhibiting the phenotype, disorganization may include oscillation of the previously aligned chromosomes. Also in some embodiments, the mitotic spindle moves but does not break.

Cells exhibiting the mp2 phenotype typically die in one of two ways after the chromosomes and microtubules become disorganized. In both cases, the cells ultimately die by what is morphologically similar to an apoptotic or mitotic catastrophe pathway. In both cases, it is only mitotic cells that die. In one case, a cell progresses to apoptosis (or a morphologically similar state) directly from mitosis. In the other case, a cell first transitions to a state where its DNA decondenses, or slips back into a 4N state (four sets of chromosomes). In some cases, the chromosomes are no longer visible in cells with decondensed DNA. From the decondensed state, a cell exhibiting the mp2 phenotype progress to apoptosis (or the similar state).

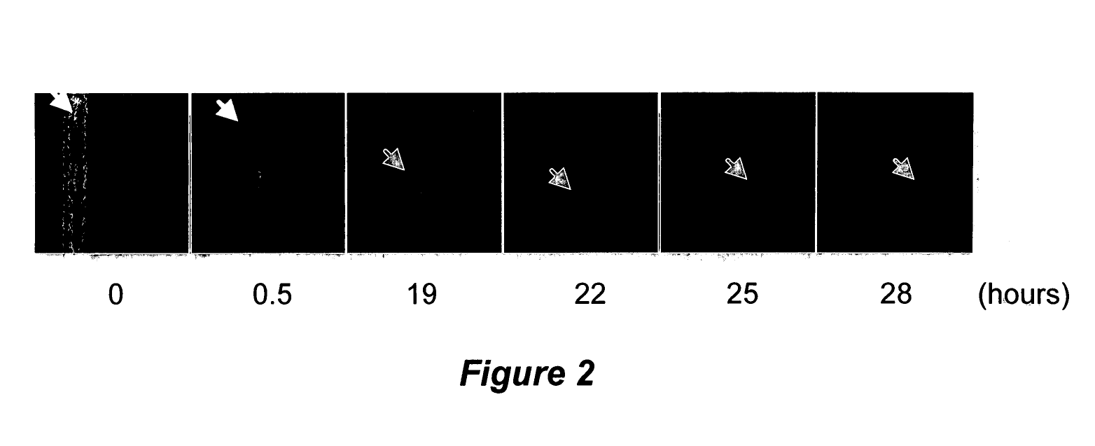

FIG. 2 shows progression of a cell that dies from apoptosis after chromosome decondensation. FIG. 2 is a time-lapse montage of GFP-histone 2B in a SKOV3 cell exhibiting the mp2 phenotype to according to certain embodiments. The cell was treated with 10 μM of an mp2 compound and imaged at 10×. Images were taken every ten minutes. Images from 0, 0.5, 19, 22, 25 and 28 hours are shown in FIG. 2 as indicated below each image. The montage shows an interphase cell just prior to condensation (0 hours). At 0.5 hours, mitosis is arrested, the cell having entered the prolonged metaphase characterized by aligned and mostly stable chromosomes at the metaphase plate. The cell stays in this state for the next 18 or so hours. At 19 hours, the condensed chromosomes have become disorganized. The image at 22 hours shows the mitotic cell just prior to decondensation, and at 25 hours the chromosomes have decondensed. Cell apoptosis is shown at 28 hours. As noted above, other cells in a population will die from mitosis without decondensation.

In many cases, a population of mp2 phenotype cells will have significant numbers of cells dying by each mechanism (e.g., about 35% of the cells die via the decondensed DNA route and about 65% die via the direct route). These modes of cell death and the relative numbers of cells dying by these two modes are characteristics that may be employed to identify cells exhibiting the mp2 phenotype. One may also characterize the number of cells that undergo decondensation relative to those that die from mitosis or complete mitosis. However, all of these effects are cell line dependent, and fixed time point experimental results can vary based on the kinetics of each cell line's doubling time and delayed mitosis.

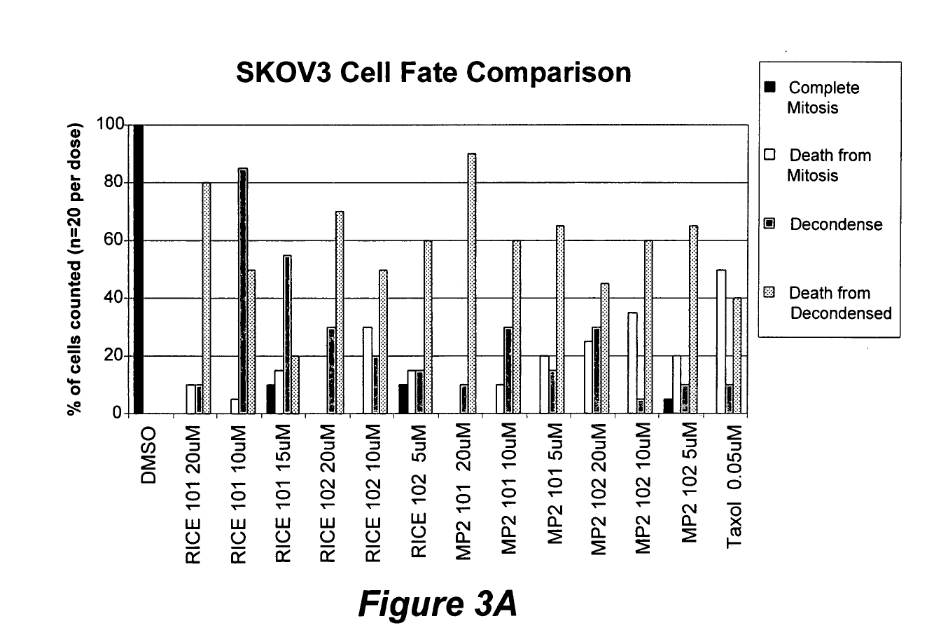

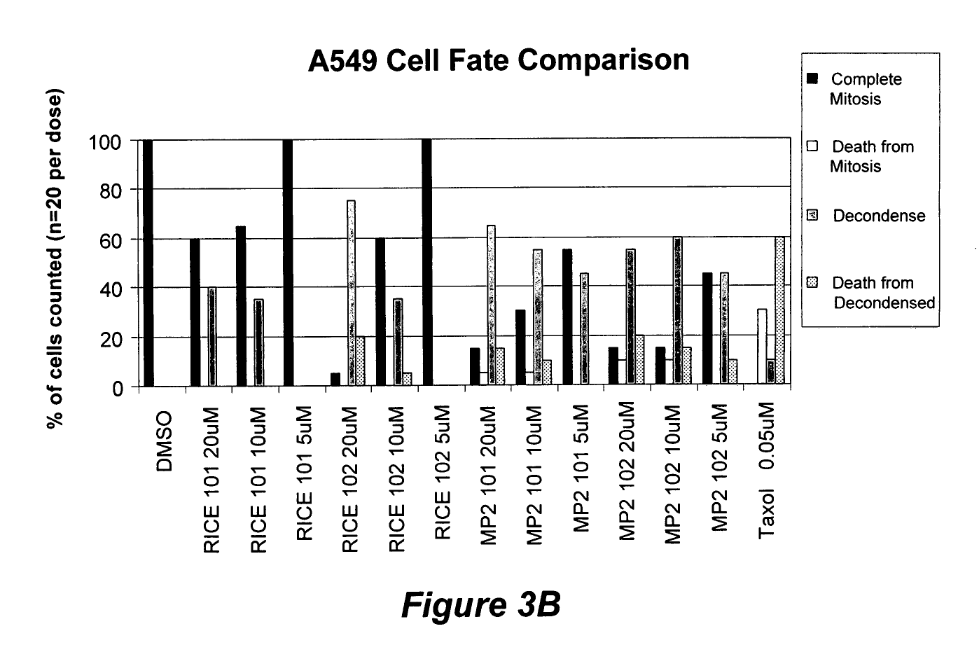

FIGS. 3a and 3b are bar charts showing the relative numbers of cells completing mitosis, undergoing apparent apoptosis directly from mitosis, undergoing decondensation (undetermined fate), undergoing decondensation prior to death for SKOV3 (FIG. 3a) and A549 (FIG. 3b) cells exhibiting the phenotype. The data used to construct the chart was obtained by time-lapse movies of cells marked with GFP-Histone 2B. Movies were collected on cell populations treated with two different mp2 stimulus compounds at various concentrations, as well as on DMSO-treated (control) cells, cells treated with 0.5 μM Taxol, and cells treated with compounds that produce a rice phenotype. (Rice compounds are another class of mitotic inhibitors. The rice phenotype and compounds that produce it are disclosed in U.S. patent application Ser. No. 11/155,934, hereby incorporated by reference.) It should be noted that decondensation (undetermined fate) refers to cells that were observed to undergo decondensation but for whose eventual fate (death or recovery) was not recorded in the time period of the movie (5-6 days after treatment). It is believed that most of these cells would eventually die.

As can be seen from FIG. 3a, most SKOV3 cells exhibiting the phenotype slip into a 4N state with fragmented DNA—the pathway depicted in FIG. 2. This profile is distinguished from Taxol, for which the highest number of cells die directly from mitosis. FIG. 3b shows the relative numbers of cells fates for A549 cells exhibiting the phenotype. As with the SKOV3 cells, very few cells die from mitosis, but undergo decondensation—in contrast to the cells treated with Taxol. Unlike the SKOV3 cells, most cells observed to decondense had not died by the end of the movie. However, it is believed that most of the cells that undergo decondensation eventually die. Regardless of the mechanism of death, FIGS. 3a and 3b show that most cells exhibiting the phenotype die.

To the extent that mp2 phenotype cells undergo apoptosis, various techniques may be employed to identify apoptotic cells. As illustrated with FIG. 2, such cells can be identified visually as those that stop moving and whose nuclei fragment. More fundamentally, apoptosis is characterized by a pathway that includes changes in certain membrane proteins, depolarization of the mitochondrial membrane, release of cytochrome C from mitochondria, activation of various caspase enzymes (caspase 3 is a major isoform involved in apoptosis), condensation, fragmentation and granularization of the nuclei, and breakdown of various nuclear and cellular proteins including actin, and microtubules. In addition, apoptotic cells become loosely attached to their substrate and can be easily dislodged. Many of these manifestations can be identified by image analysis. Examples include exposure of phosphatidyl serines on membrane proteins, the migration of cytochrome c from the mitrochondria into other regions of the cell, changes of mitochondrial membrane potential, activation of caspase 3, cleavage of caspase substrates (PARP, microtubule and actin), and condensation, fragmentation and granularization of the nuclei.

Another property of cells undergoing apoptosis is that they tend to become loosely attached to a substrate. Both cytoplasm shrinkage and loss of attachment is probably a result of cytoskeleton damage by caspases. This property can be detected by exposing the culture to a treatment that will tend to dislodge and remove loosely attached cells. One way to accomplish this is by carefully washing a cell culture under consideration. The level of apoptosis has been found to correlate well to a “washout coefficient” based on cell counts in washed and unwashed cultures exposed to a stimulus suspected of inducing apoptosis; e.g., (cc (unwashed)—cc(washed))/cc(unwashed).

A more detailed discussion of various techniques for identifying apoptotic cells is presented in U.S. patent application Ser. No. 10/623,486 (U.S. Patent Publication No. 20050014216), filed Jul. 18, 2003, by Mattheakis et al., and titled “PREDICTING HEPATOTOXICITY USING CELL BASED ASSAYS,” and U.S. patent application Ser. No. 10/719,988 (U.S. Patent Publication No. 20050014216), 20050014217, filed Nov. 20, 2003, by Mattheakis et al., and titled “PREDICTING HEPATOTOXICITY USING CELL BASED ASSAYS,” both of which are incorporated herein by reference in their entireties and for all purposes.

Frequently, cells exhibiting the mp2 phenotype present unique features only during mitosis. During interphase, the phenotypic features of mp2 and control cells may be essentially indistinguishable. That is, only minimal phenotypic differences occur between control and mp2 phenotype cells during interphase, at least with respect to certain components of interest such as tubulin, DNA, and Golgi.

This behavior suggests that the target of the compounds that are eliciting the mp2 phenotype are specific for a protein or proteins that are only used by the cell in mitosis and are specific for those targets. This is similar to inhibition of the mitotic kinesin KSP. Other compounds that arrest cells in mitosis via targets that are also used during interphase (for example Taxol, Vincristine, and Vinblastine which target microtubules) show clear morphological effects on interphase and are predicted to have much lower therapeutic indexes in the human body.

Generally, in order to characterize the interphase phenotype of a cell or cell population, one must first determine whether a cell is in an interphase stage. Mitotic and interphase cells can be distinguished by analyzing various particular cellular features. For example, the signal from a marker for a phosphorylated histone may be used for this purpose. As indicated, one example of such marker is a marker for phospho-histone 3 (PH3) such an anti-phospho-histone 3 (PH3) antibody coupled to a fluorophore. If PH3 staining is not available, or desirable, then cells can be classified as mitotic or interphase based on a combination of the size of nuclei and the amount of DNA material in nuclei (as revealed by DNA staining using DAPI or Hoechst stains). After each cell, or image object, has been classified as interphase or mitotic, the mitotic and interphase phenotypes can be characterized.

The phenotype of the interphase cells may be characterized in terms of a wide variety of cellular features. Such features can relate to nuclear or cellular morphology, e.g., size, area, shape metrics, branching, etc. Cellular features relating to measures of the total amount of a component of a cell can be used, e.g. the total tubulin, total actin, total Golgi apparatus and other measures, often derived from measurements of the total intensity of radiation captured from a particular component of a cell. Also, measures of the texture of a cellular image can be used and which relate to physical properties of components of cells. Still other cellular features relating to various types of generic cellular phenomena can be related to the interphase phenotype, such as changes in growth rate, cytoskeletal organization, alterations in organization and functioning of the endocytotic pathway, changes in expression and/or localization of transcription factors, receptors and the like. One, some or all of those cellular features can be considered in characterizing the interphase phenotype. It is expected that these features in mp2 interphase cells would be similar to those of normal (non-tumor) cells exposed to an mp2 stimulus.

In one specific example, a particular group of cellular features for characterizing the interphase phenotype of a cell could include, for all cells that are not mitotic:
the average size of cell nuclei;
the average elliptical axis ratio for nuclei;
the average kurtosis of intensity of cells Golgi;
the average pixel intensity for Golgi apparatus in cells;
the average cell area;
the elliptical axis ratio for cells;
the form factor (area divided by perimeter) for cells;
the kurtosis of the intensity of tubulin;
the second moment of a cell's tubulin intensity;
the average total intensity of tubulin for each cell;
the proportion of branched (i.e. having projections) cells.

In this example, the above group of cellular features constitutes the group of cellular features, which in combination define the interphase phenotype signature. A sub-group of these features can be used, or alternatively other groups of cellular features can be used. As will be appreciated, there are a large number of variables in this group of features. Some of these variables may be more important than others, i.e., may be more affected by a treatment than others. The combination of these features can be thought of as defining a vector in a multivariate space (defined by the cellular features) and which is characteristic of the interphase phenotype.

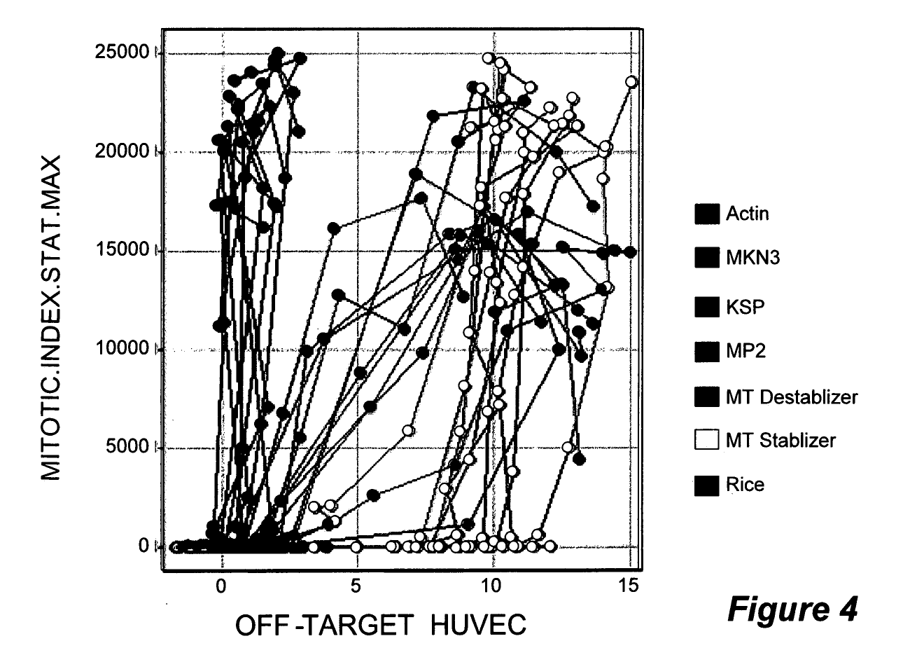

In one embodiment, after each cellular feature has been characterized, and similarly for the control group cellular features, a distance in multivariate space may be calculated. This can be the distance from a normal interphase phenotype as presented in the horizontal axis of FIG. 4 (described below). For the purposes of simplicity of discussion, if it is assumed that there are only three cellular features (a, b, c) comprising the interphase phenotype signature, and where the subscript ‘t’ refers to a feature of a treated cell and the subscript ‘c’ refers to a feature of a control cell, then the distance (L₁) in multivariate space between the interphase signature of the treated cells and interphase signature of the control cells can be calculated as L₁=|aₜ-a_{c}|+|bₜ-b_{c}|+|cₜ-c_{c}|, which provides the interphase metric.

Alternatively, the Euclidean distance (L₂) can be calculated using L₂=√((aₜ-a_{c}) ²+(bₜ-b_{c})²+(cₜ-c_{c})²) to provide the interphase metric. Other methods of calculating the separation in multivariate space between the treated cell interphase signature and the control cell interphase signature can also be used. Note that any of the various methods described in this section may be employed to similarly measure distance between multivariate signatures of chromatin observed in mitotic cells that potentially exhibit the phenotypes of this invention.

In treatments other than those producing the mp2 phenotype, one may commonly observe, in the interphase cells, a breakdown of the actin cytoskeleton of a cell, or the Golgi apparatus. This breakdown may be a more or a less dominant effect of the treatment than mitotic breakdown. Regardless, such effects will result in a relatively large separation distance from the control phenotype for interphase cells.

FIG. 4 presents data showing that certain compounds producing the mp2 phenotype have very little effect on phenotypic features of normal interphase cells. In FIG. 4, the vertical axis presents a mitotic index statistic that measures a compound's ability to cause mitotic arrest in SKOV3 tumor cells (and thereby its ability to have a profound effect on the phenotype of mitotic cells), and the horizontal axis presents a “combined distance” from a normal interphase phenotype in normal HUVEC cells. The combined distance takes into account various features that characterize interphase phenotype, including those described above (i.e., the average size of cell nuclei, the average elliptical axis ratio for nuclei, the average kurtosis intensity of cells, etc.) Greater values on the horizontal axis indicate greater deviations from a control phenotype for interphase cells. Lines connect increasing concentrations for one compound

The mitotic index statistic along the vertical axis FIG. 4 is the mitotic index in SKOV3 cells, while the distance from the normal interphase phenotype data presented along the horizontal axis was generated from treating HUVEC interphase cells with the listed compounds and measuring the number of standard deviations of the above listed features from HUVEC interphase cells treated in DMSO.

As explained, many stimuli that have a significant impact on mitosis also have some clearly defined impact on interphase features. This is exactly what is observed with known microtubule destabilizers, such as Taxol. Note that the microtubule destabilizer data (the orange data points in FIG. 4) extends well into the region on the right side of the plot where the interphase phenotype is widely separated from the control interphase phenotype. However, the compounds that produce the mp2 phenotype (the light blue data points in FIG. 4) have minimal impact on interphase features—while having a significant impact on mitotic index. This is illustrated by the fact that all data points for these compounds lie on the left side of the plot in FIG. 4. In this analysis, when the distance value is less than about 5, a compound is generally considered to have little effect on interphase cells. The mp2 compounds in FIG. 4 have similar effects on interphase cells as do inhibitors of mitotic kinesins KSP and MKN3.

In addition to the multivariate distance charted in FIG. 4, a normal interphase phenotype may be characterized in terms of the individual features and/or a profile of individual features as compared to a control phenotype.

5. Tumor-Normal Differential Sensitivity

As discussed above, the normal interphase aspect of the phenotype indicates that stimuli inducing the phenotypes affect mitotic-specific proteins only and thus have few or no off-target effects. A related feature of the phenotype is the tumor-normal differential sensitivity—the phenotype is induced in tumor cells but not in normal cells, or has a significantly reduced effect on these cells. In some cases, the tumor-normal differential sensitivity of mp2 stimulus compounds is greater than other known mitotic inhibitors.

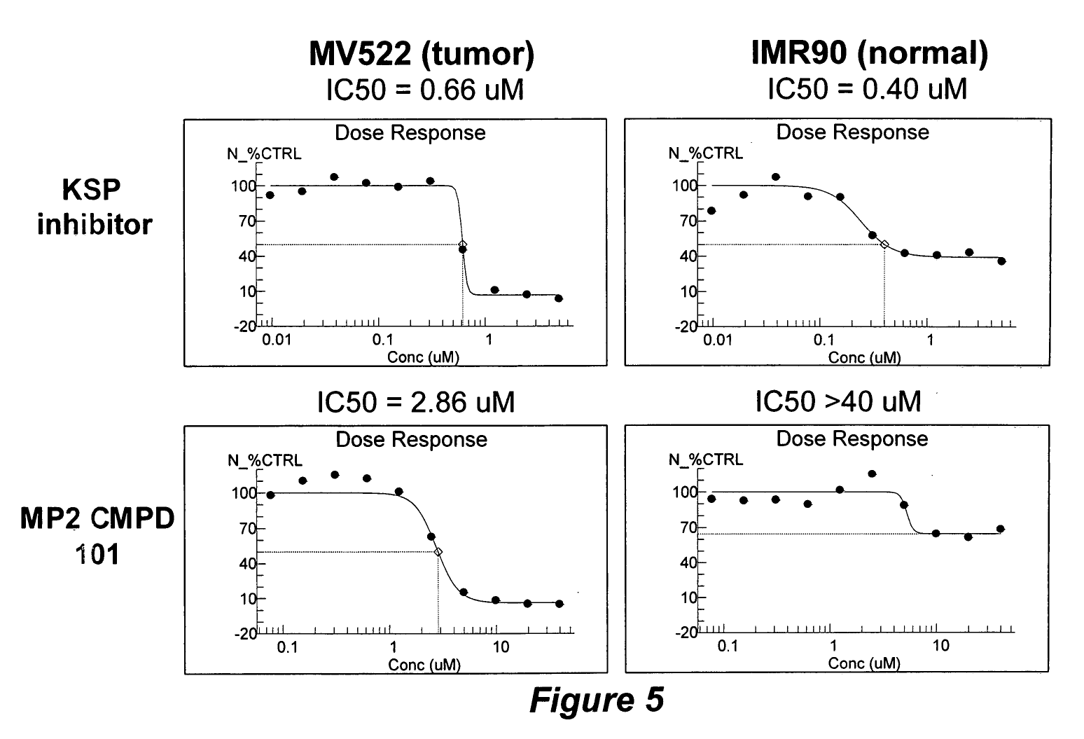

FIG. 5 shows IC50 dose response curves for a KSP inhibitor and an mp2 compound in a tumor cell line (MV522) and a normal cell (IMR90). IC50 (the concentration at which growth is inhibited 50%) data is one way in which the tumor-normal differential sensitivity may be expressed. The KSP inhibitor (a known mitotic inhibitor) shows little tumor-normal differential sensitivity (an IC50 of 0.66 μM for the tumor cell and 0.40 μM for the normal cell). The mp2 compound, however, shows an over thirteen-fold increase in IC50 for the normal cell lines. IC50 differentials of 50-fold indicate that normal cells are highly resistant to mitotic inhibitors and therefore unwanted side effects are likely to be less pronounced.

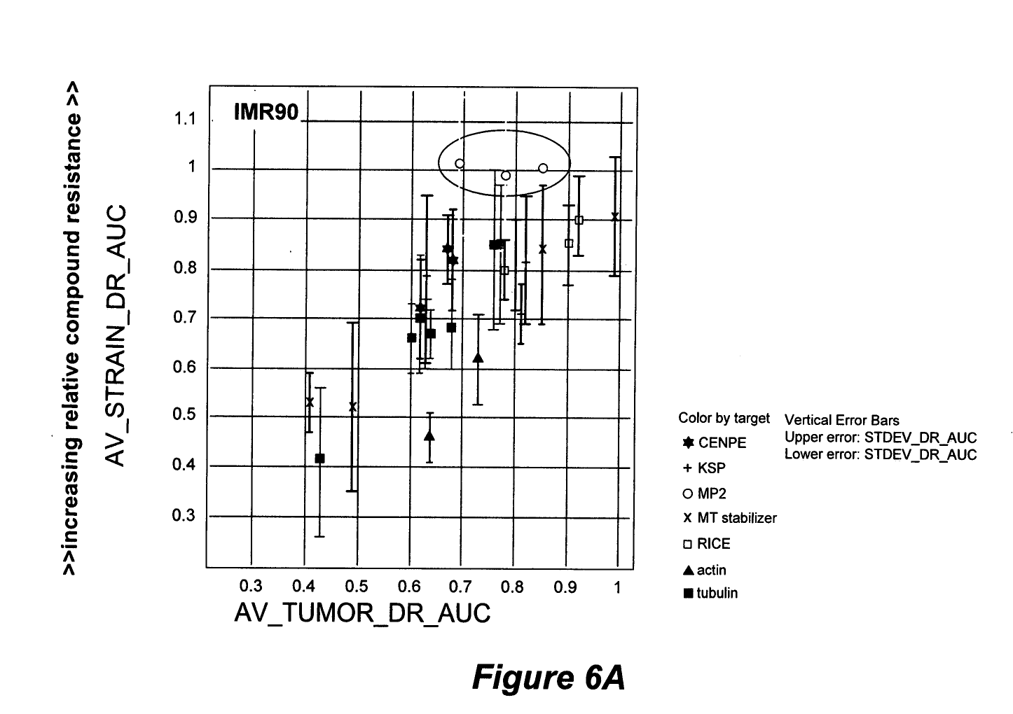

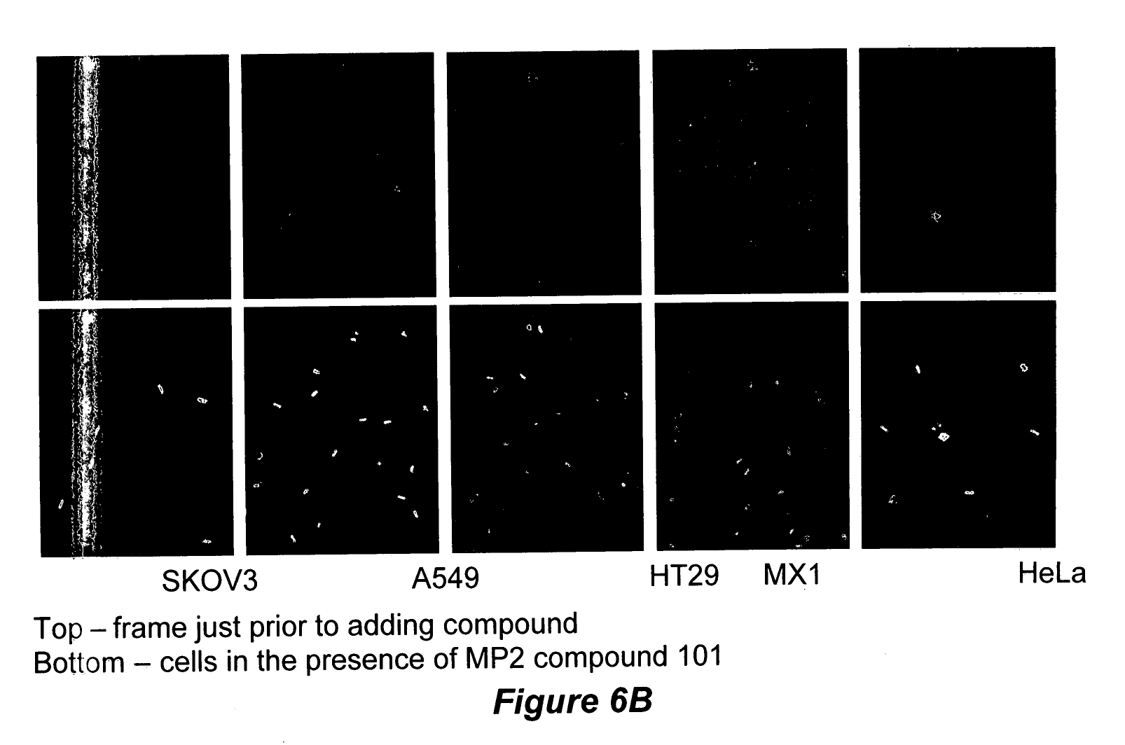

Another way of expressing tumor-normal differential sensitivity is based on the area under the dose response curve (AUC), with larger AUC values indicating higher resistance. FIG. 6a shows AUC data for mitotic inhibitors for normal (IMR90) and tumor (HMEC) cells. The AUC for the IMR90 cell line is presented under the y-axis, a measure of increasing relative compound resistance, while the x-axis shows the average AUC values for a panel of tumor cell lines (SKOV3, A549, HT29, MV522) treated with various mitotic inhibitors. The yellow data points show that three mp2 compounds are more resistant to IMR90 cells than all of the other mitotic inhibitors tested, i.e., microtubule stabilizers and destabilizers, rice compounds and inhibitors of KSP and CENP-E.

6. Penetrance

In some embodiments, the phenotype may be further characterized by its penetrance, i.e. the percent of cells exposed to a stimulus that exhibit the phenotype. The penetrance is highly dependent on both cell line and strength or concentration of the stimulus. However, in certain embodiments, all of cells in a population exhibit the phenotype that enter mitosis will exhibit the phenotype.

7. Cell Line Specific Response to Stimuli that Induce the Phenotype

Compounds that induce the phenotype are effective against a wide range of cancer cell lines. FIG. 6b shows images of the mp2 phenotype on five cancer cells types: SKOV3, A549, HT29, MX1 and HeLa cells. The images show GFP-histone 2B; the top images were taken just prior to adding an mp2 inducing compound and the bottom images show cells in the presence of the compound. Cells in each bottom image are exhibiting mitotic arrest.

Stimuli that induce the mp2 phenotype do so in a cell line specific manner; although the mp2 phenotype is observed across cancer cell lines, compounds that induce the phenotype have greater potency in inducing the phenotype and/or inhibiting growth against some cell lines than others. The cell line specificity of the mp2 phenotype can be considered unique. Many compounds that promote mitotic arrest also show cell line specificity, but at varying degrees for different cell types. The NCI (National Cancer Institute) measures the sensitivity of 60 cell lines to a wide panel of therapeutic agents (dtp.nci.nih.gov/dtpstandard/dwindex/index.jsp) and that data shows that compounds can be classified by the pattern of their sensitivity, and that a compound, like Taxol, can have over 3 orders of magnitude in potency differences between cell types. A compound can thus be uniquely described by its cell line specificity pattern, such that any compound with that pattern may induce the same phenotype

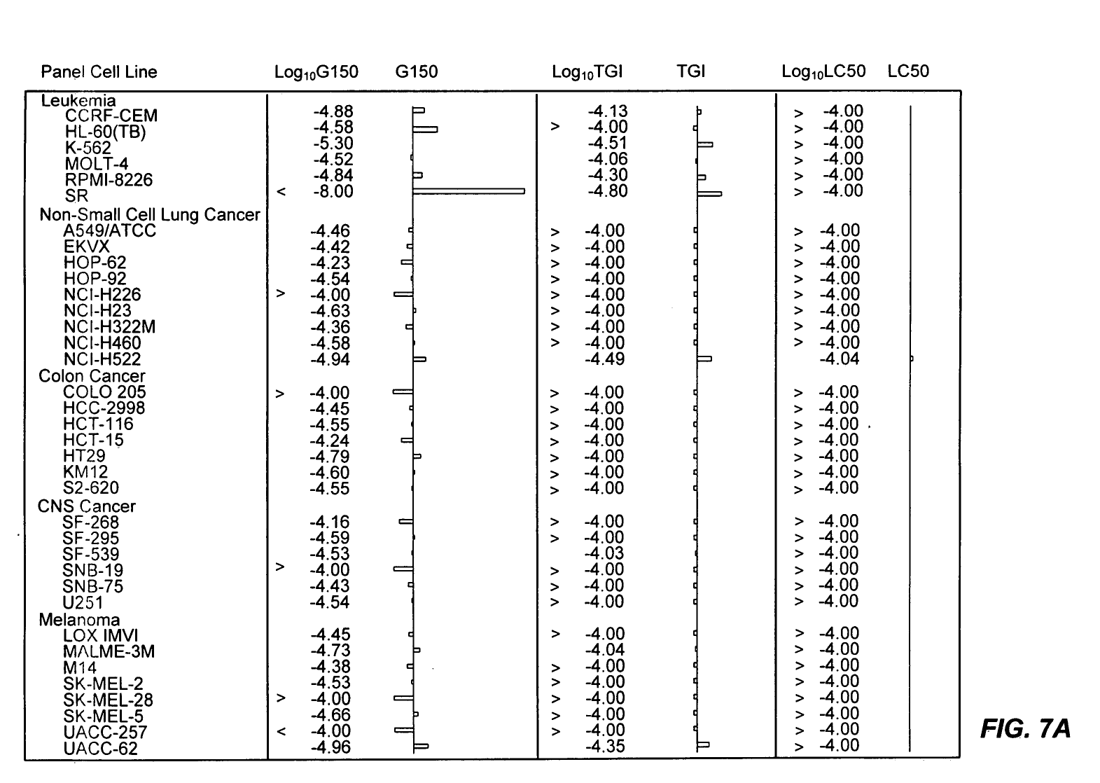

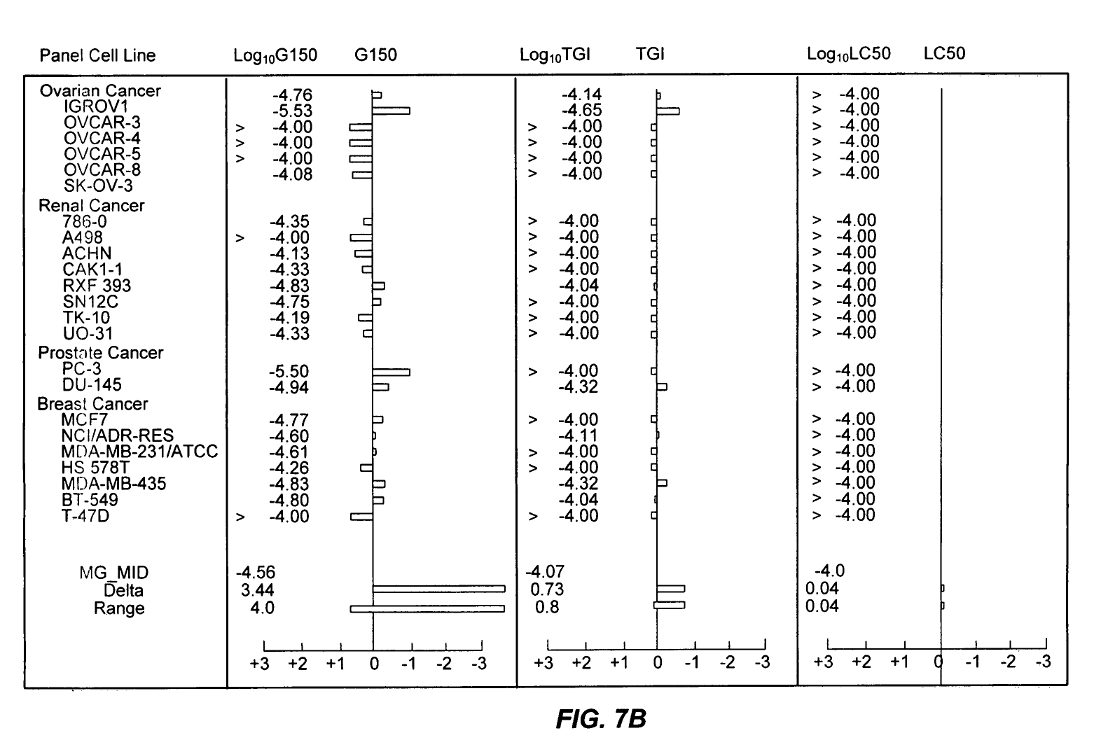

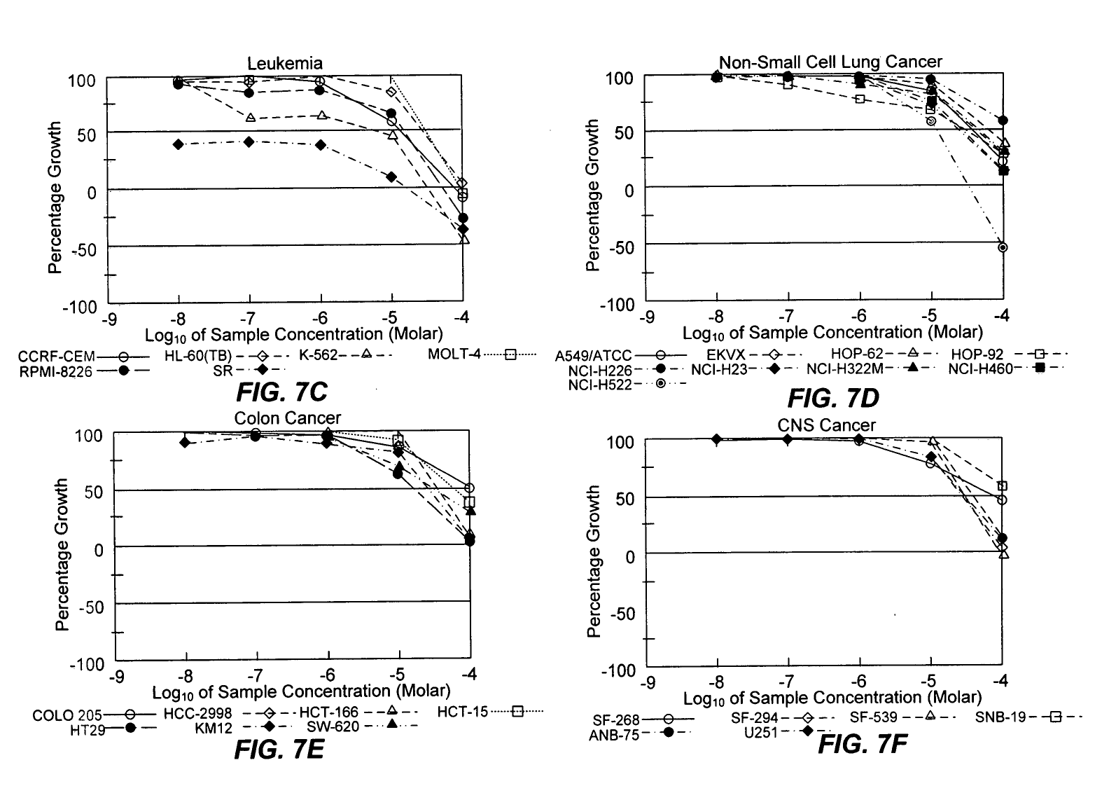

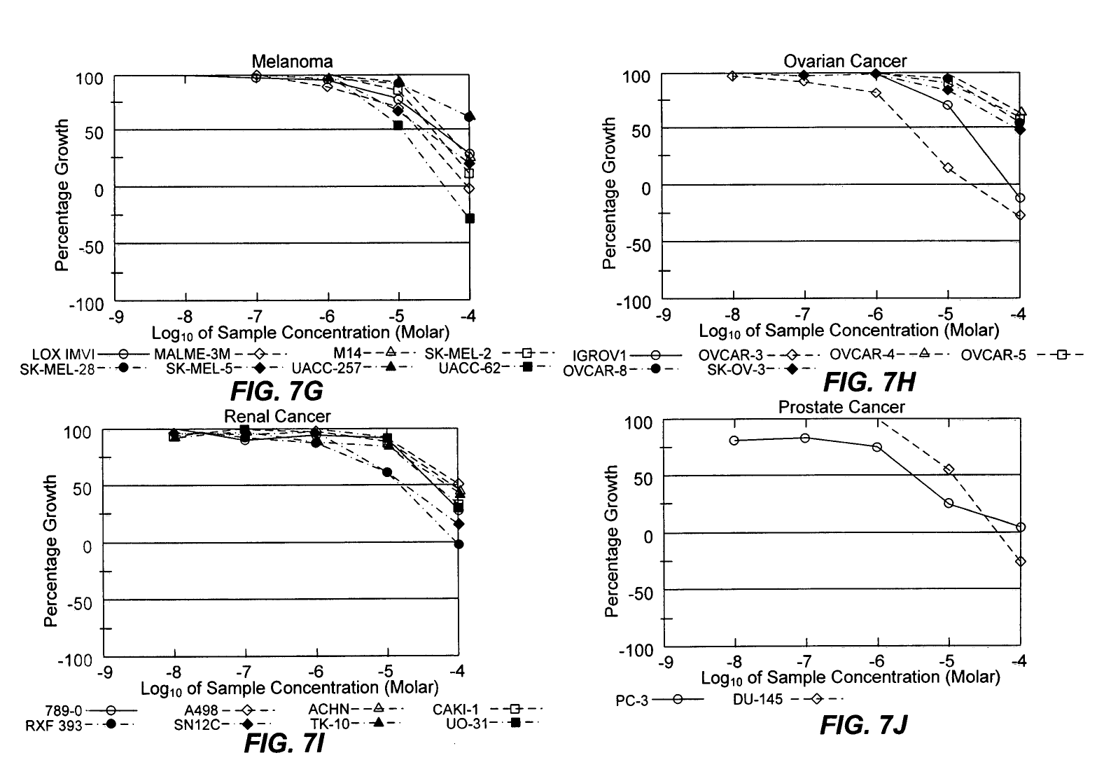

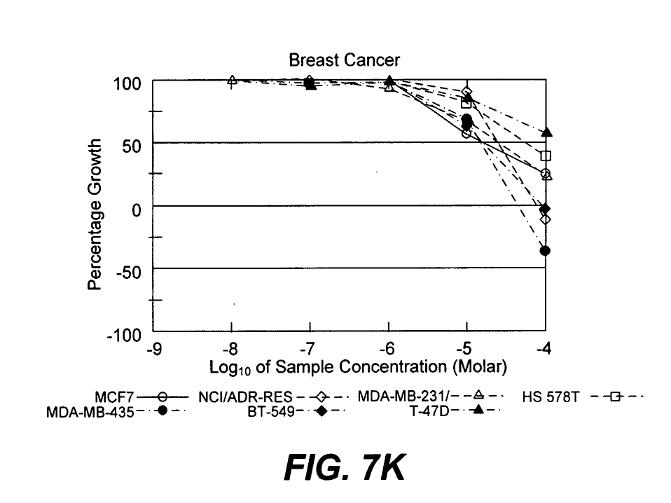

As an example, FIGS. 7A and 7B shows an NCI pattern for an mp2 stimulus compound, specifically growth inhibition (G150), tumor growth inhibition (TGI) and lethal concentration (LC50) data. It should be noted that the pattern shown in FIGS. 7A and 7B was not found to have a statistically significant match to any of the patterns of any of the known compounds in the NCI database.

FIGS. 7C through 7K presents the data as percentage growth as a function of compound concentration, with the data separated by types of cancer cells. Notably, the percentage growth decreases by close to or more than 50% for all cancer cells tested for the maximum test concentration of 10⁻⁴ molar. These figures show that the compound has significantly greater effect on MDA-MB-435 breast cancer cells than on T-47D breast cancer cells at the concentrations tested.

In some embodiments, the phenotype may be characterized by the correlation of the mp2 stimulus compound with the growth inhibition pattern shown in FIGS. 7A through 7K. Compounds that have about 90% correlation to growth inhibition pattern shown in FIGS. 7A through 7K would be expected to produce the mp2 phenotype. Compounds producing the mp2 phenotype have a low correlation to known compounds and classes of compounds in the NCI database. The highest correlation of any known compound or class of compounds in the NCI database to the pattern shown in FIGS. 7A and 7B was found to be only about 70%.

8. Low Trailing Resistance

As discussed above with reference to FIGS. 3a and 3b, a high percentage of cells in a population exhibiting the phenotype die, typically in a defined manner. This is in contrast to cells treated with some mitotic inhibitors, which recover after mitosis is arrested. Cells treated with these mitotic inhibitors may exhibit residual viability reduced but persistent growth at high concentrations of a compound. One way this is quantified is the trailing resistance of a compound. Trailing resistance is a measure of the residual viability after treatment with a compound in a clonogenic viability assay. Trailing resistance has been shown to correlate with in vivo resistance to inhibitors of KSP in xenographic models.

The phenotypes of the present invention may be further characterized by the trailing resistance of the cells after the other features of the phenotype are induced in a cell or cells in a population of cells. Specifically, the cells show little or no trailing resistance.

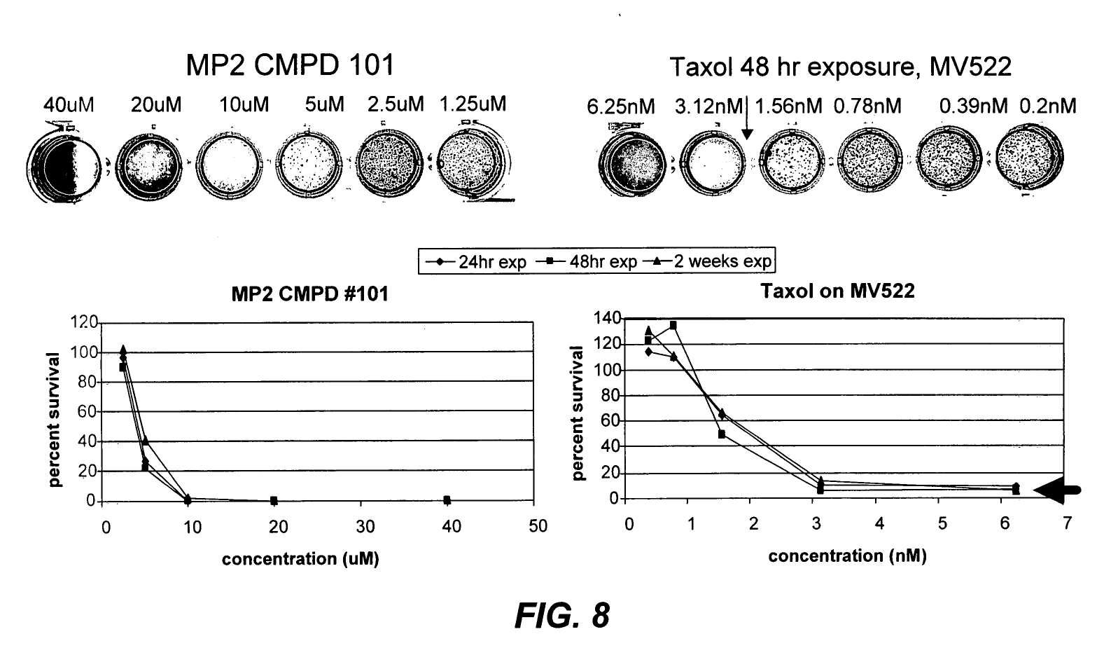

Stimuli inducing the mp2 phenotype have little or no trailing resistance as shown in clonogenic viability assays. FIG. 8 shows results of clonogenic viability assays of MV522 cells treated with an mp2 producing compound and Taxol. Images from the clonogenic viability assay showing cell cultures after 48 hours exposure to various concentrations are also shown. Unlike Taxol and other known mitotic inhibitors, the mp2 compound does not induce trailing resistance in MV522 cells.

Another measure of the residual viability is the percent survival at in the presence of stimulus at five times greater concentration than the IC50. MV522 cells have less than 10% survival in the presence of mp2 compounds—lower than inhibitors of the KSP and CENP-E. This predicts that compounds that induce the mp2 phenotype are highly effective in in vivo tumor cells. However, trailing resistance is cell line dependent. For example, the percent survival of COLO205 cells by this measure is higher for the mp2 compounds tested than for other types of mitotic inhibitors tested.

IV. Experimental Protocol

An experiment to determine whether a treatment can produce the mp2 phenotype can be carried out in many ways. Frequently it will involve one or more assay plates. An assay plate is typically a collection of wells arranged in an array with each well holding at least one cell or a related group or population of cells which have been exposed to a treatment or which provides a control group, population or sample. In other embodiments, multi-well plates are not used and single sample holders can be used. As explained above, a treatment can take many forms and in one embodiment can be a particular drug or any other external stimulus (or a combination of stimuli and/or drugs) to which cells are exposed on an assay plate or have previously been exposed. Experimental protocols for investigating the effect of a treatment will be apparent to a person of skill in the art and can include variations in the dose level, incubation time, cell type, cell line, marker set and other parameters, which are typically varied as part of an experimental protocol. After the cells have been treated, the extent of the effect of the treatment for producing the mp2 phenotype is evaluated by investigating, typically in a quantitative way, how the properties of the cells that are involved in or related to the mp2 phenotype have changed.

For example, the phenotypic feature of interest could be congression and alignment of chromosomes during mitosis. After the treatment has been applied to the cells and features have been extracted from captured images, then some of the cellular features can be used to classify cells as interphase or mitotic. As previously explained, the amount of fluorescence from an anti-phospho-histone 3 (PH3) coupled to a fluorophore can be used to distinguish between mitotic and interphase cells. After each cell, or image object, has been classified as interphase or mitotic (or discarded as being an imaging artifact), a characterization of mitotic chromatin can be made. The effect of the treatment can then be determined by comparing this characterization for the treated cells with the same characterization for a control group of cells.

As explained, there will likely be other cellular features of cell components which are involved in or relate to mp2 phenotype and which will also be affected by the treatment and so change. Therefore using a one or a combination of the relevant phenotypic features, the effect of the treatment can be evaluated.

In addition to merely determining whether a given treatment produces the mp2 phenotype of this invention, the investigation may study different dose levels of the stimulus (or stimuli) in question. It has been found that different dose levels and experimental protocols can result in different relevant phenotypic features arising. Significantly different dose levels may be required to produce the mp2 phenotypic features in different cell lines.

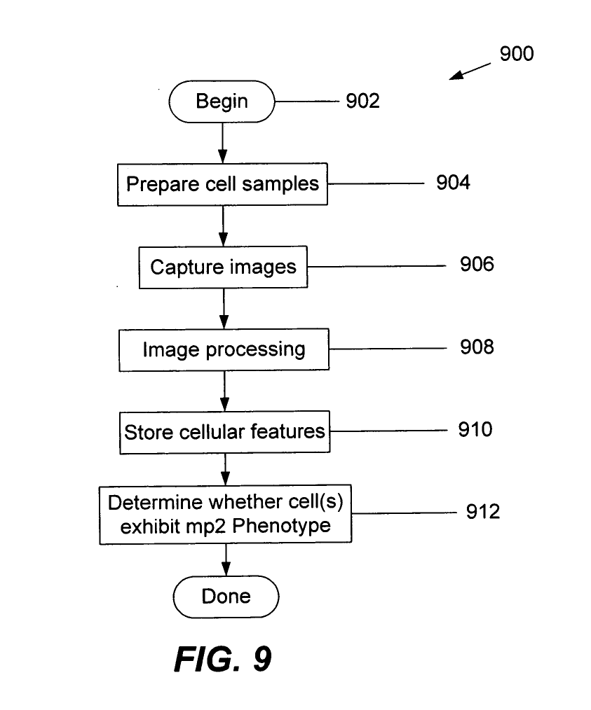

Having discussed the overall methodology of the invention, an example embodiment will now be described in greater detail in the context of an image based collection of cellular features. FIG. 9 shows a flow-chart 900 illustrating an example of the general method and illustrating various aspects of the invention. The method begins at 902 and at a step 904 cell samples are prepared for investigation.

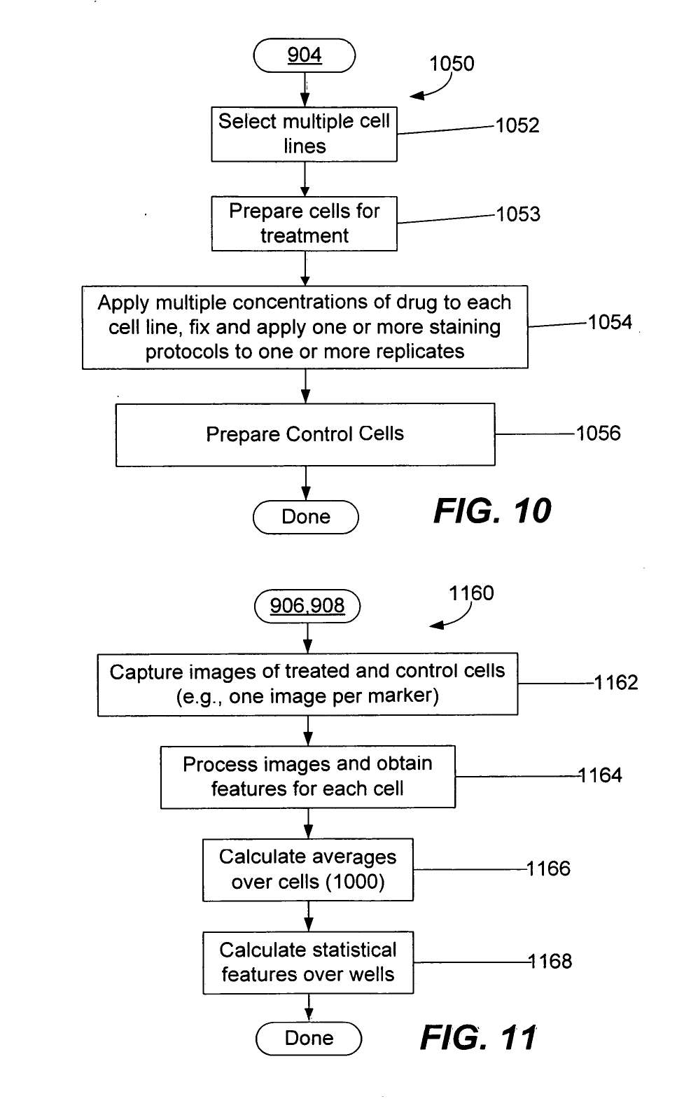

FIG. 10 shows a flow chart 1050 illustrating a number of cell sample preparation steps that can be carried out in one embodiment, giving an example of one suitable experimental protocol, and corresponding generally to step 904. Not all the activities and operations illustrated in FIG. 10 are essential. Some operations may be omitted and other operations may be added. The details of each operation may be varied depending on the particular experiment being carried out.

Although illustrated as sequential in FIG. 10, steps 1054 and 1056 do not need to be carried out in sequence and can be carried out in parallel, independently of each other. In a first step 1052, a particular one or a plurality of different cell types are selected. In the embodiment described, six cell lines for the particular cell type are selected although fewer or more cell lines can be used. In one embodiment, the cell lines used are A549, DU145, SKOV3 A498, HUVEC and SF268. Next, in a step 1053, the cells are prepared by, for example, plating them on appropriate substrates. At a step 1054, the treatment is applied to the cells. Well plates can be used to hold the cells and a population of cells from a single cell line is provided in each separate well arranged over a well plate or a number of well plates.

In the illustrated embodiment, at step 1054, the cells are treated, chemically fixed, and stained. However, this is not necessary and in another embodiment, live cells can be used which express a fluorescent protein or stained with live dyes and so no fixing or staining operations are required. In greater detail, wells are provided holding a population of cells. The treatment, in this example a compound, to be investigated is applied to the cells at different concentration levels, by dilution in culture medium. In one example, eight different concentration or dose levels are used, with a different dose level in each well. Fewer or more dose levels can be used as appropriate. The experiment is replicated three times so as to provide three sets of results for each concentration level. Fewer replicates can be used based on cost considerations, but larger numbers of replicates are preferred as providing data with a lower noise level. The drug and cells can be allowed to incubate for a fixed period of time, e.g. in one embodiment 24 hours, to allow the treatment to take effect. In other embodiments, the cells are allowed to incubate for varying periods of time, in order to investigate the time variation of the treatment. The cells can then be chemically fixed, for a single time point assay. The cells for each cell line are subject to a first staining protocol and a second staining protocol, which may involve multiple stains depending on the number and type of cellular features to be marked. Hence, in the described embodiment, 288 wells (eight dose levels, six cell lines, two staining protocols and three replicates) are used each holding a cellular population or group therein.

At the same time as the treated cells are being prepared, a number of control populations of cells are also prepared in step 1056. Preparation techniques for control cells will be different depending on the drug formulation. The cells are subject to the same staining treatments, fixation and incubation periods as the treated cells, but without being subjected to the treatment. In one embodiment, the cells are incubated with DMSO, at the same percentage levels as that used to administer the treatments, in order to provide controls for each cell line and staining or experimental condition. In one embodiment eight control wells are provided on each well plate. This provides at least one control for each cell line/staining protocol combination. Hence the cell sample preparation step 904 results in eight treatment concentrations, in triplicate, with cells stained according to two different protocols, and for six different cell lines and with control populations of cells which have not been exposed to the treatment. It is not necessary to use more than one stain or staining protocol and in other embodiments a single stain only can be used.

Returning to FIG. 9, the cellular features can be obtained from the cells using an image capture and processing technique. At step 906, images of the cells are captured and at step 908 various imaging processing operations are carried out and cellular features are derived from the captured images of the cells. Once all the desired the cellular features have been obtained from the images, or derived from other cellular features, then the cellular features are stored for future use in the evaluation of the mp2 phenotype at a step 910. In another embodiment, the cellular features are used straight away to determine whether the mp2 phenotype has been produced and then discarded. In another embodiment steps 908 and 910 are bypassed and the images are manually evaluated. In other words, the mp2 phenotype can be identified qualitatively without steps 908 and 910

FIG. 11 shows a flow chart 1160 illustrating the image capture 906, processing and feature extraction 908 steps of flow chart 900 in greater detail. At a first step 1162, images of the cell populations in each well are captured. In this example, images are captured for each of the eight concentration levels, in triplicate for each cell line and for both of the staining protocols. Similarly, images are captured for each of the groups of control cells for each cell line and for both staining protocols. In particular, a first image or set of images is captured of each well for the stains used in the first staining protocol and then a second image or group of images for each well is captured for the stains used in the second staining protocol. One or more images can be captured for each well and/or each stain.

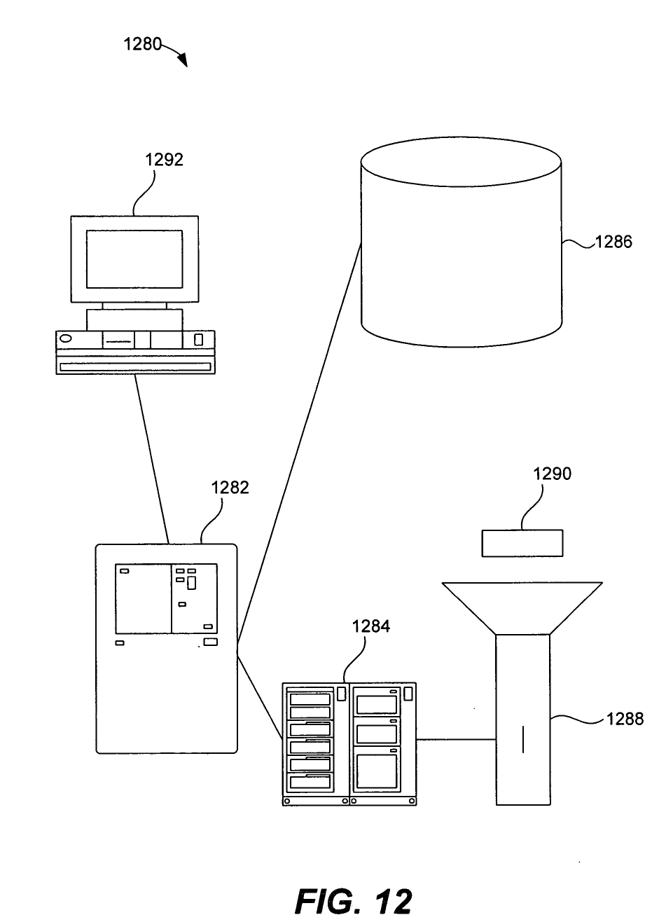

FIG. 12 shows a schematic block diagram of an image capture and image processing system 1280 which can be used to capture and process the images of cells or cell parts during steps 906 and 908 and store the cellular features in step 910. This diagram is merely an example and should not limit the scope of the claims herein. One of ordinary skill in the art would recognize other variations, modifications, and alternatives. The present system 1280 includes a variety of elements such as a computing device 1282, which is coupled to an image processor 1284 and is coupled to a database 1286. The image processor receives information from an image-capturing device 1288, which includes an optical device for magnifying images of cells, such as a microscope. The image processor and image-capturing device can collectively be referred to as the imaging system herein. The image-capturing device obtains information from a plate 1290, which includes a plurality wells providing sites for groups of cells. These cells can be cells that are living, fixed, cell fractions, cells in a tissue, and the like. The computing device 1282 retrieves the information, which has been digitized, from the image-processing device and stores such information into the database 1286.

A user interface device 1292, which can be a personal computer, a work station, a network computer, a personal digital assistant, or the like, is coupled to the computing device. In the case of cells treated with a fluorescent marker, a collection of such cells is illuminated with light at an excitation frequency from a suitable light source (not shown). A detector part of the image-capturing device is tuned to collect light at an emission frequency. The collected light is used to generate an image, which highlights regions of high marker concentration.

Sometimes corrections can be made to the measured intensity. This is because the absolute magnitude of intensity can vary from image to image due to changes in the staining and/or image acquisition procedure and/or apparatus. Specific optical aberrations can be introduced by various image collection components such as lenses, filters, beam splitters, polarizers, etc. Other sources of variability may be introduced by an excitation light source, a broadband light source for optical microscopy, a detector's detection characteristics, etc. Even different areas of the same image may have different characteristics. For example, some optical elements do not provide a “flat field.” As a result, pixels near the center of the image have their intensities exaggerated in comparison to pixels at the edges of the image. A correction algorithm may be applied to compensate for this effect. Such algorithms can be developed for particular optical systems and parameter sets employed using those imaging systems. One simply needs to know the response of the systems under a given set of acquisition parameters.

After the images have been captured, at step 1164, the captured images are processed using any suitable image processing and image correction techniques in order to extract the cellular features for the cells from the stored captured images.

A number of image processing steps can be carried out in step 1164 and not all the steps described are essential. Certain steps may be omitted and other steps may be added depending on the exact nature of the image capture process and markers used. The image can be corrected to remove any artifacts introduced by the image capture system and to remove any background. Other conventional image correction techniques, which will improve the quality of the image can also be used. Typically, one chooses nuclear markers and cytoplasmic markers which generate radiation at different wavelengths in order to allow capture of separate nuclear images and cytoplasmic images. Therefore different image correction techniques may be used for the nuclear and cytoplasm images, or for images captured of different markers or stains. Similarly, in the rest of the processes, different techniques may be used for the nuclear and cytoplasmic images, depending on the markers used. Also, different processing techniques can be carried out depending on the type of imaging that is used, e.g. brightfield, confocal or deconvolution.

After image correction, a segmentation process is carried out on the images in order to identify individual objects or entities within the image. Any suitable segmentation process may be used in order to obtain various cellular objects or components, such as nuclear and cellular objects and components. Typically nuclear DNA markers provide a strong signal and there is a high contrast in the image and an edge detection based segmentation process can be used. For segmenting cells, a watershed type method can be used instead. The segmentation process typically identifies edges where there is a sudden change in intensity of the cells in the image and then looks for closed connected edges in order to identify an object. Segmentation will not be described in greater detail as it is well understood in the art and so as not to obscure the present invention. As indicated above, exemplary segmentation procedures are described in U.S. Patent Publications Nos. US-2002-0141631-A1 and U.S.-2002-0154798-A1.

Additional operations may be performed prior to, during, or after the imaging operation 906 of FIG. 9. For example, “quality control algorithms” may be employed to discard image data based on, for example, poor exposure, focus failures, foreign objects, and other imaging failures. Generally, problem images can be identified by abnormal intensities and/or spatial statistics.

In a specific embodiment, a correction algorithm may be applied prior to segmentation to correct for changing light conditions, positions of wells, etc. In one example, a noise reduction technique such as median filtering is employed. Then a correction for spatial differences in intensity may be employed. In one example, the spatial correction comprises a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc.

Generally the images used as the starting point for the methods of this invention are obtained from cells that have been specially treated and/or imaged under conditions that contrast the cell's marked components from other cellular components and the background of the image. Typically, the cells are fixed and then treated with a material that binds to the components of interest and shows up in an image (i.e., the marker).

At every combination of dose, cell line and staining protocol, one or more images can be obtained. As mentioned, these images are used to extract various parameter values of cellular features of relevance to a biological, phenomenon of interest. Generally a given image of a cell, as represented by one or more markers, can be analyzed, in isolation or in combination with other images of the same cell (as provided by different markers), to obtain any number of image features. These features are typically statistical or morphological in nature. The statistical features typically pertain to a concentration or intensity distribution or histogram.

The various phenotypic features of the mp2 phenotype have been described above, together with techniques for identifying these features. The image analysis methods of this invention identify such features and possibly others. Some general feature types suitable for detection or quantification with this invention include a cell, or nucleus where appropriate, count, an area, a perimeter, a length, a breadth, a fiber length, a fiber breadth, a shape factor, a elliptical form factor, an inner radius, an outer radius, a mean radius, an equivalent radius, an equivalent sphere volume, an equivalent prolate volume, an equivalent oblate volume, an equivalent sphere surface area, an average intensity, a total intensity, an optical density, a radial dispersion, and a texture difference. These features can be average or standard deviation values, or frequency statistics from the parameters collected across a population of cells. In some embodiments, the features include features from different cell portions or cell lines.

After the features have been extracted from the image (1164) they are stored (910) in database 1286, and analysis of the features is carried out in order to assess the effect of the treatment on the cells.

As explained above, some of the cellular features obtained for the cells are simple features, e.g. the area of a nucleus. Other cellular features are statistical in nature, e.g. the standard deviation of the nuclear area for a group of cells, and reflect properties of the group of cells in a well or related wells. It will be appreciated that any simple or complex cellular feature than can be derived from the images is suitable for use in the present invention and that the invention is not to be limited to the specific examples given, nor to the specific sequence of actions, which is merely by way of an illustrative example. The result of step 1164 can be thousands or tens of thousands of cellular features derived from each of the treated wells and control wells.

In general in steps 1166 and 1168 cells from a well are evaluated and some statistics for that well, e.g. the average of a property, are calculated. Then, the same quantity is obtained for the replicate wells (e.g., the other five wells when the experiment is replicated six times) statistics are computed on those statistics for the replicate wells in order to aggregate (e.g., obtain the median of the average value mentioned above). However, averaging is not necessary and instead cell level information can be used, and have all further computations to be based on cell level information. Hence, for each compound/cell line/time point/marker set/etc there would be thousands of data points.

At step 1166, at each dose level and for each cell line, the cellular features can be averaged, e.g. to obtain an average nuclear area for the cells from a certain cell line at a certain dose level. Hence an average simple cellular feature can be obtained for each cell line at each dose level. However, it is not necessary to calculate averages over cells. Also, other statistical measures can be used such as the median, specific quantiles, standard deviations and other measures of the statistical properties of a group of objects. Further, the statistical properties need not be calculated over all cells, but can be calculated over a sub-population of cells, for example over the sub-group of interphase cells. In that case, a cell cycle related classification of the cells is carried out prior to summarizing or averaging the cell feature values.

At step 1168, more complex cellular features, based on a statistical analysis of the properties of the cells in the wells, rather than the properties of a single cell, are calculated over all the wells for each cell line at each dose level. Hence the cellular features obtained characterize the simple cellular features and statistical cellular features for the cellular populations at each dose level for each cell line.

In other embodiments, the simple cellular features and the statistical cellular features can be determined across cell lines so as to be characteristic of the effect of the treatment across different cell lines. In other embodiments, different incubation times can be used for a given concentration and the cellular features can be averaged over the different incubation times in order to provide cellular features characteristic of the effect of the treatment at the same dose level but over different incubation times.

Returning to FIG. 9, after the cellular features have been calculated and stored, at step 910 a quantitative measure of the presence or absence of the mp2 phenotype may be calculated based on the cellular features. See step 912.

Some embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer (e.g., computer 1282). The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

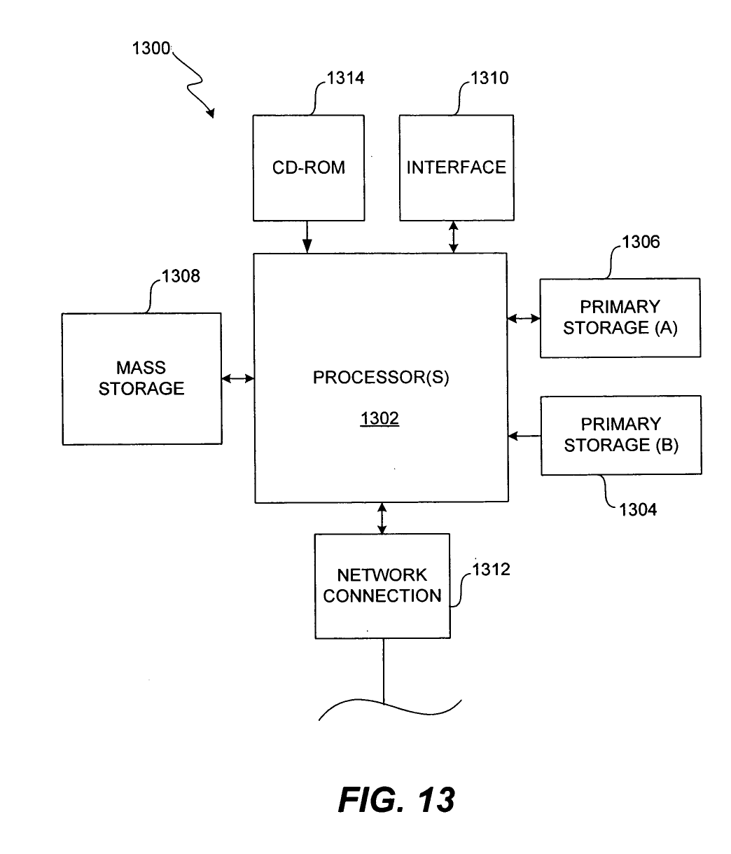

FIG. 13 illustrates a typical computer system that, when appropriately configured or designed, can serve as an image analysis apparatus of this invention. The computer system 1300 includes any number of processors 1302 (also referred to as central processing units, or CPUs) that are coupled to storage devices including primary storage 1306 (typically a random access memory, or RAM), primary storage 1304 (typically a read only memory, or ROM). CPU 1302 may be of various types including microcontrollers and microprocessors such as programmable devices (e.g., CPLDs and FPGAs) and unprogrammable devices such as gate array ASICs or general purpose microprocessors. As is well known in the art, primary storage 1304 acts to transfer data and instructions uni-directionally to the CPU and primary storage 1306 is used typically to transfer data and instructions in a bi-directional manner. Both of these primary storage devices may include any suitable computer-readable media such as those described above. A mass storage device 1308 is also coupled bi-directionally to CPU 1302 and provides additional data storage capacity and may include any of the computer-readable media described above. Mass storage device 1308 may be used to store programs, data and the like and is typically a secondary storage medium such as a hard disk. It will be appreciated that the information retained within the mass storage device 1308, may, in appropriate cases, be incorporated in standard fashion as part of primary storage 1306 as virtual memory. A specific mass storage device such as a CD-ROM 1314 may also pass data uni-directionally to the CPU.

CPU 1302 is also coupled to an interface 1310 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1302 optionally may be coupled to an external device such as a database or a computer or telecommunications network using an external connection as shown generally at 1312. With such a connection, it is contemplated that the CPU might receive information from the network, or might output information to the network in the course of performing the method steps described herein.

In one embodiment, the computer system 1300 is directly coupled to an image acquisition system such as an optical imaging system that captures images of cells. Digital images from the image generating system are provided via interface 1312 for image analysis by system 1300. Alternatively, the images processed by system 1300 are provided from an image storage source such as a database or other repository of cell images. Again, the images are provided via interface 1312. Once in the image analysis apparatus 1300, a memory device such as primary storage 1306 or mass storage 1308 buffers or stores, at least temporarily, digital images of the cells. In addition, the memory device may store the quantitative phenotypes that represent the points on the response path. The memory may also store various routines and/or programs for analyzing the presenting the data, including the phenotype characterization and image presentation. Such programs/routines may include programs for performing principal component analysis, regression analyses, path comparisons, and for graphically presenting the response paths.

VI. Other Embodiments

Although the above has generally described the present invention according to specific processes and apparatus, the present invention has a much broader range of applicability. In particular, the present invention has been described in terms of cellular phenotypes that are derived primarily from image analysis, but is not so limited, as the phenotypic characterizations presented herein may also be derived in whole or in part by techniques other than image analysis. Of course, those of ordinary skill in the art will recognize other variations, modifications, and alternatives.

What is Claimed is:

1. An mp2 phenotype embodied in a mammalian cell or a population of mammalian cells, wherein the mp2 phenotype comprises:
a) mitotic arrest characterized by (i) chromosomes well-aligned at the metaphase plate; and (ii) chromosome residence time at the metaphase plate substantially longer than that of a control cell or cell population.

2. The phenotype of claim 1, further comprising:
b) during interphase, the cell or population of cells exhibits a phenotype that is substantially similar to that of the control cell or cell population.

3. The phenotype of claim 1, further comprising:
b) chromosomes that congress to the metaphase plate in a time and manner substantially similar to that of a control cell or cell population.

4. The phenotype of claim 1, wherein the mitotic arrest is further characterized by stable microtubule-kinetochore alignment at the metaphase plate.

5. The phenotype of claim 1, further comprising:
b) a higher percentage of the cells in the cell population that die prematurely in comparison to the control cell or cell population.

6. The phenotype of claim 1, wherein the cell or cells in the population of cells die by apoptosis.

7. The phenotype of claim 1, wherein mitotic arrest lasts from about 3-24 hours.

8. The phenotype of claim 1, wherein the chromosome residence time at the metaphase plate is at least about three times longer than that of the control cell or cell population.

9. The phenotype of claim 1 wherein 1-5 chromosome pairs oscillate at the metaphase plate during mitotic arrest.

10. The phenotype of claim 1 further comprising:
b) a higher percentage of cells have chromosomes that undergo DNA decondensation in comparison to the control cell or cell population.

11. The phenotype of claim 1 wherein non-tumor cells are less susceptible to stimuli that produce the mp2 phenotype than tumor cells.

12. The phenotype of claim 1, wherein, during interphase, the mp2 phenotype is substantially similar to the control phenotype in terms of one or more of the following: cytoskeletal organization, cell shape, alterations in organization and functioning of the endocytic pathway, and changes in expression or localization of transcription factors or receptors.

13. A mammalian cell or cell population having an mp2 phenotype, wherein the mp2 phenotype comprises:
a) mitotic arrest characterized by (i) chromosomes well-aligned at the metaphase plate; and (ii) a chromosome residence time at the metaphase plate substantially longer than that of a control cell or cell population.

14. The mammalian cell or cell population of claim 13, wherein the mp2 phenotype is produced by applying a stimulus to the mammalian cell or cell population while the cell or cell population does not exhibit the mp2 phenotype in order to induce a transformation to produce the mp2 phenotype.

15. The mammalian cell or cell population of claim 14, wherein applying the stimulus comprises administering a compound to the mammalian cell or cell population while the cell or cell population does not exhibit the mp2 phenotype.

16. The mammalian cell or cell population of claim 13, wherein the mp2 phenotype exhibits during interphase a phenotype that is substantially similar to that of the control cell or cell population.

17. The mammalian cell or cell population of claim 13, wherein the mp2 phenotype further comprises, in the cell or some cells in the population of cells, chromosomes that congress to the metaphase plate in a time and manner substantially similar to that of a control cell or cell population.

18. The mammalian cell or cell population of claim 13, wherein the mitotic arrest is further characterized by stable microtubule-kinetochore alignment at the metaphase plate.

19. The mammalian cell or cell population of claim 13, wherein the mp2 phenotype further comprises a higher percentage of the cells in the cell population that die prematurely in comparison to the control cell or cell population.

20. The mammalian cell or cell population of claim 13, wherein mitotic arrest lasts from about 3-24 hours.

21. The mammalian cell or cell population of claim 13, wherein the chromosome residence time at the metaphase plate is at least about three times longer than that of the control cell or cell population.

22. A method of determining whether a stimulus produces a transformation associated with an mp2 phenotype, the method comprising:
a) exposing a mammalian cell or mammalian cell population to the stimulus;
b) allowing the stimulus to interact with the cell or cell population in a manner that transforms a normal phenotype in susceptible cells to the mp2 phenotype, wherein the mp2 phenotype has at least the following features: mitotic arrest characterized by (i) chromosomes well-aligned at the metaphase plate; and (ii) a chromosome residence time at the metaphase plate substantially longer than that of a control cell or cell population;
c) imaging the cell or cell population to capture features that characterize the phenotype of the cell or cell population; and
d) analyzing the image to determine whether the cell or cell population exhibits the phenotypic features specified in (b), to thereby determine whether the stimulus produces the transformation.

23. The method of claim 22, wherein the stimulus is a chemical compound.

24. The method of claim 23, wherein imaging the cell or cell population comprises capturing multiple images in a time-lapse manner.

25. The method of claim 22, wherein the mp2 phenotype further comprises: during interphase, the cell or population of cells exhibits a phenotype that is substantially similar to that of the control cell or cell population.

26. The method of claim 22, wherein the mp2 phenotype further comprises chromosomes that congress to the metaphase plate in a time and manner substantially similar to that of the control cell or cell population.

27. The method of claim 22, wherein the mitotic arrest is further characterized by stable microtubule-kinetochore alignment at the metaphase plate.

28. The method of claim 22, wherein the mp2 phenotype further comprises a higher percentage of the cells in the cell population that die prematurely in comparison to the control cell or cell population.

29. The method of claim 22, wherein the mp2 phenotype further comprises a higher percentage of the cells in the cell population that die in comparison to the control cell or cell population.

30. The method of claim 22, wherein compounds that produce the mp2 phenotype in tumor cells do so to a significantly less degree in non-tumor cells.

31. The method of claim 22, wherein (b)-(d) comprise a clonogenic viability assay.

32. A method of characterizing a mammalian cell or a mammalian cell population on the basis of its phenotype, the method comprising:
a) receiving data characterizing the phenotype of the cell or cell population;
b) analyzing the data to determine whether the cell or cell population possesses the following features mitotic arrest characterized by (i) chromosomes well-aligned at the metaphase plate; and (ii) chromosome residence time at the metaphase plate substantially longer than that of a control cell or cell population; and
c) characterizing the cell or cell population as having a mp2 phenotype when the cell or cell population is found to possess at least the features specified in (b).

33. The method of claim 32, wherein the data characterizing the phenotype of the cell or cell population comprises data specifying whether the cell or cell population has been exposed to a stimulus that interacts with a target associated with the mp2 phenotype.

34. The method of claim 32, wherein (b) further comprises analyzing the data to determine whether the cell or cell population possesses one or more of the following additional features: (a) during interphase the cell or population of the interphase cells exhibits a phenotype that is substantially similar to that of an interphase control cell or the interphase cells of the control cell population; (b) chromosomes that congress to the metaphase plate in a time and manner substantially similar to that of the control cell or cell population; (c) a higher percentage of the cells in the cell population that die in comparison to the control cell or cell population; and
(d) stimuli that produce the mp2 phenotype do so selectively in tumor cell lines.

35. The method of claim 32, wherein the cell or cells in the population of cells die by apoptosis upon reaching a mitotic state.

36. The method of claim 35, wherein some of the cells that die by apoptosis do so after their DNA decondenses.

37. A computer program product comprising a machine readable medium on which is provided program code for characterizing a mammalian cell or a mammalian cell population on the basis of its phenotype, the program code comprising:
a) code for receiving data characterizing the phenotype of the cell or cell population;
b) code for analyzing the data to determine whether the cell or cell population possesses the following features: mitotic arrest characterized by (i) chromosomes well-aligned at the metaphase plate and (ii) a chromosome residence time at the metaphase plate substantially longer that of a control cell or cell population; and
c) code for characterizing the cell or cell population as having a mp2 phenotype when the cell or cell population is found to possess at least the features specified in (b).

38. The computer program product of claim 37, wherein (b) further comprises code for analyzing the data to determine whether the cell or cell population possesses the following additional features: (a) during interphase the cell or population of the interphase cells exhibits a phenotype that is substantially similar to that of an interphase control cell or the interphase cells of the control cell population; (b) chromosomes that congress to the metaphase plate in a time and manner substantially similar to that of the control cell or cell population; (c) a higher percentage of the cells in the cell population that die in comparison to the control cell or cell population; and (d) during interphase the cell or population of the interphase cells exhibits a phenotype that is substantially similar to that of an interphase control cell or the interphase cells of a control cell population;

39. An apparatus for characterizing a mammalian cell or a mammalian cell population on the basis of its phenotype, the apparatus comprising:
a) means for receiving data characterizing the phenotype of the cell or cell population;
b) means for analyzing the data to determine whether the cell or cell population possesses the following features: mitotic arrest characterized by (i) chromosomes well-aligned at the metaphase plate, (ii) a chromosome residence time at the metaphase plate substantially longer that of a control cell or cell population; and
c) means for characterizing the cell or cell population as having an mp2 phenotype when the cell or cell population is found to possess at least the features specified in (b).
